SEQUENCE LISTING (14 sequences)

SEQ ID NO: 1 - DNA, 297 bp, Homo sapiens
atgaacaaacacttcttgttcctcttcctcctttactgcctcattgtggcagtgacatcacttcagtgcataacatgccaccttcgcacacggacagaccgctgtagaagaggctttggtgtctgtactgctcagaagggcgaggcatgcatgctcttaaggatttaccagcgcaatactctccagatatcatacatggtgtgtcagaaattctgcagagacatgacatttgatctcaggaatcggacttatgttcatacatgctgcaactacaattactgtaactttaaactctaa

SEQ ID NO: 2 - DNA, 288 bp, Homo sapiens
atgaacacactgctccttgtgagcttatcttttctctacctcaaagaggttatgggtctgaagtgtaatacctgcatatacacagaaggatggaagtgtatggcaggccgaggcacttgcattgcaaaagaaaatgagttatgttcaacaacagcctatttcaggggagacaaacatatgtactcaacacatatgtgtaagtataagtgccgggaagaggagtcctccaaaagaggcctgttgagagtgacactgtgctgtgacagaaacttctgtaatgtcttctaa

SEQ ID NO: 3 - DNA, 342 bp, Homo sapiens
atgcttgttctctttctcctgggcacagtctttctgctctgcccatattggggtgaacttcatgaccctataaaagcgactgaaataatgtgttatgaatgtaaaaaatatcatcttgggttatgctatggtgtcatgacatcctgctccctgaagcataaacagtcctgtgcagttgagaacttttacatccttacaaggaaagggcagagcatgtatcattattcaaaactgtcgtgtatgaccagctgtgaggacatcaacttcctggggttcacgaagagggtagagctcatctgttgtgatcatagtaactactgcaacctccctgagggagtttag

SEQ ID NO: 4 - DNA, 318 bp, Homo sapiens
atgcttgttctctttctcctgggcacagtctttctgctctgcccatattgggcgactgaaataatgtgttatgaatgtaaaaaatatcatcttgggttatgctatggtgtcatgacatcctgctccctgaagcataaacagtcctgtgcagttgagaacttttacatccttacaaggaaagggcagagcatgtatcattattcaaaactgtcgtgtatgaccagctgtgaggacatcaacttcctggggttcacgaagagggtagagctcatctgttgtgatcatagtaactactgcaacctccctgagggagtttag

SEQ ID NO: 5 - DNA, 171 bp, Homo sapiens
atgaacacactgctccttgtgagcttatcttttctctacctcaaagagggagacaaacatatgtactcaacacatatgtgtaagtataagtgccgggaagaggagtcctccaaaagaggcctgttgagagtgacactgtgctgtgacagaaacttctgtaatgtcttctaa

SEQ ID NO: 6 - DNA, 213 bp, Homo sapiens
atgcttgttctctttctcctgggcacagtctttctgctctgcccatattggggtgaacttcatgaccctataaaagggcagagcatgtatcattattcaaaactgtcgtgtatgaccagctgtgaggacatcaacttcctggggttcacgaagagggtagagctcatctgttgtgatcatagtaactactgcaacctccctgagggagtttag

SEQ ID NO: 7 - DNA, 189 bp, Homo sapiens
atgcttgttctctttctcctgggcacagtctttctgctctgcccatattgggggcagagcatgtatcattattcaaaactgtcgtgtatgaccagctgtgaggacatcaacttcctggggttcacgaagagggtagagctcatctgttgtgatcatagtaactactgcaacctccctgagggagtttag

SEQ ID NO: 8 - protein, 101 aa, Homo sapiens
MNKHFLFLFLLYCLIVAVTSLQCITCHLRTRTDRCRRGFGVCTAQKGEACMLLRIYQRNTLQISYMVCQKFCRDMTFDLRNRTYVHTCCNYNYCNFKLKEY

SEQ ID NO: 9 - protein, 95 aa, Homo sapiens
MNTLLLVSLSFLYLKEVMGLKCNTCIYTEGWKCMAGRGTCIAKENELCSTTAYFRGDKHMYSTHMCKYKCREEESSKRGLLRVTLCCDRNFCNVF

SEQ ID NO: 10 - protein, 113 aa, Homo sapiens
MLVLFLLGTVFLLCPYWGELHDPIKATEIMCYECKKYHLGLCYGVMTSCSLKHKQSCAVENFYILTRKGQSMYHYSKLSCMTSCEDINFLGFTKRVELICCDHSNYCNLPEGV

SEQ ID NO: 11 - protein, 105 aa, Homo sapiens
MLVLFLLGTVFLLCPYWATEIMCYECKKYHLGLCYGVMTSCSLKHKQSCAVENFYILTRKGQSMYHYSKLSCMTSCEDINFLGFTKRVELICCDHSNYCNLPEGV

SEQ ID NO: 12 - protein, 56 aa, Homo sapiens
MNTLLLVSLSFLYLKEGDKHMYSTHMCKYKCREEESSKRGLLRVTLCCDRNFCNVF

SEQ ID NO: 13 - protein, 70 aa, Homo sapiens
MLVLFLLGTVFLLCPYWGELHDPIKGQSMYHYSKLSCMTSCEDINFLGFTKRVELICCDHSNYCNLPEGV

SEQ ID NO: 14 - protein, 62 aa, Homo sapiens
MLVLFLLGTVFLLCPYWGQSMYHYSKLSCMTSCEDINFLGFTKRVELICCDHSNYCNLPEGV

(19) United States
(12) Patent Application Publication
(10) Pub. No.: US 2010/0196351 A1
(43) Pub. Date: Aug. 5, 2010

(54) SECRETED PATE-LIKE PROTEINS

(75) Inventor: Daniel H. Wreschner, Efrata (IL)

Correspondence Address: THE NATH LAW GROUP, 112 South West Street, Alexandria, VA, 22314, US

(73) Assignee: GENESWITCH INNOVATIONS LLC, Waban, MA (US)

(21) Appl. No.: 12/449,586

(22) PCT Filed: Feb. 14, 2008
(86) PCT No.: PCT/IL2008/000201

§ 371 Date: Apr. 12, 2010

Related U.S. Application Data
(60) Provisional application No. 60/901,336, filed on Feb. 15, 2007.

(51) Int. Cl. A61K 39/395; C07H 21/04; C07K 14/00; A61K 38/16; C12Q 1/68; A61P 25/28

(52) U.S. Cl. 424/130.1; 536/23.5; 530/350; 530/324; 514/12; 435/6

(57) ABSTRACT

Novel PATE-like polynucleotides and polypeptides, are provided. Also described are splice-variants resulting in smaller polypeptides including five cysteine residues and capable of forming PATE-like polypeptide multimerse. The described polynucleotides were found to have a surprising expression profile, predominantly in the reproduction system and nervous system. Further provided are pharmaceutical compositions and methods for treatment of various diseases, in particular nervous system disorders, more specifically Alzheimer's disease.

### FIELD OF THE INVENTION

This invention relates to secreted polypeptides expressed predominantly in prostate, testis, and brain, and to pharmaceutical compositions for the treatment of various diseases, in particular Alzheimer's disease.

### LIST OF REFERENCES

The following references are considered to be pertinent for the purpose of understanding the background of the present invention:
1. Bera, T. K., Maitra, R., Iavarone, C., Salvatore, G., Kumar, V., Vincent, J. J., Sathyanarayana, B. K., Duray, P., Lee, B. K. & Pastan, I. (2002) Proc Natl Acad Sci USA 99, 3058-63.
2. Fry, B. G., Wuster, W., Kini, R. M., Brusie, V., Khan, A., Venkataraman, D. & Rooney, A. P. (2003) J Mol Evol 57, 110-29.
3. Ploug, M. & Ellis, V. (1994) FEBS Lett 349, 163-8.
4. Ploug, M. (2003) Curr Pharm Des 9, 1499-528.
5. Tsetlin, V. I. & Hucho, F. (2004) FEBS Lett 557, 9-13.
6. Tremeau, O., Lemaire, C., Drevet, P., Pinkasfeld, S., Ducancel, F., Boulain, J. C. & Menez, A. (1995) J Biol Chem 270, 9362-9.
7. Kolbe, H. V., Huber, A., Cordier, P., Rasmussen, U. B., Bouchon, B., Jaquinod, M., Vlasak, R., Delot, E. C. & Kreil, G. (1993) J Biol Chem 268, 16458-64.
8. Adermann, K., Wattler, F., Wattler, S., Heine, G., Meyer, M., Forssmann, W. G. & Nehls, M. (1999) Protein Sci 8, 810-9.
9. Tsuji, H., Okamoto, K., Matsuzaka, Y., Iizuka, H., Tamiya, G. & Inoko, H. (2003) Genomics 81, 26-33.
10. Chimienti, F., Hogg, R. C., Plantard, L., Lehmann, C., Brakch, N., Fischer, J., Huber, M., Bertrand, D. & Hohl, D. (2003) Hum Mol Genet 12, 3017-24.
11. Freemerman, A. J., Flickinger, C. J. & Herr, J. C. (1995) Mol Reprod Dev 41, 100-8.
12. Kumar, S., Tamura, K. & Nei, M. (2004) Brief Bioinform 5, 150-63.
13. Notredame, C., Higgins, D. G. & Hering a, J. (2000) J Mol Biol 302, 205-17.
14. Soler-Garcia, A. A., Maitra, R., Kumar, V., Ise, T., Nagata, S., Beers, R., Bera, T. K. & Pastan, I. (2005) Reproduction 129, 515-24.
15. Herr, J. C., Wright, R. M., John, E., Foster, J., Kays, T. & Flickinger, C. J. (1990) Biol Reprod 42, 377-82.
16. Luo, C. W., Lin, H. J. & Chen, Y. H. (2001) J Biol Chem 276, 6913-21.
17. Coronel, C. E., Winnica, D. E., Novella, M. L. & Lardy, H. A. (1992) J Biol Chem 267, 20909-15.
18. Satomi, Y., Shimonishi, Y. & Takao, T. (2004) FEBS Lett 576, 51-6.
19. Southan, C., Cutler, P., Birrell, H., Connell, J., Fantom, K. G., Sims, M., Shaikh, N. & Schneider, K. (2002) Proteomics 2, 187-96.
20. Foster, J. A., Klotz, K. L., Flickinger, C. J., Thomas, T. S., Wright, R. M., Castillo, J. R. & Herr, J. C. (1994) Biol Reprod 51, 1222-31.
21. Emes, R. D., Beatson, S. A., Ponting, C. P. & Goodstadt, L. (2004) Genome Res 14, 591-602.
22. WO 02/102985
23. Altschul, et al., Nature Genet., 6: 119, 1994.
24. Liu, Q. and Wu J. (2006) Acta Pharmacologica Sinica 27 (10) 1277-1286.

### BACKGROUND OF THE INVENTION

A functional genomic approach has succeeded in identifying a gene coding for a secreted protein having preferential prostate and testis expression, designated the PATE gene (1; 22). The PATE protein is comprised of ten cysteine residues, with the C-terminal cysteine residue positioned within a cysteine-asparagine (CN) dipeptide sequence. The distribution of cysteine residues conforms to a consensus pattern of cysteines found in a large protein domain family of three-fingered proteins (TFP), characterized by a distinct disulfide bonding pattern between eight or ten cysteine residues. This domain is additionally found in uPAR and murine Ly-6 GPI-anchored proteins, and is also called an Ly-6/uPAR domain (2, 3). Interestingly the TFP architecture is seen also in the TGFI3 receptor family of proteins including BMP2 and activin receptors (4).

A large protein family encompassing an extensive group of GPI-anchored, transmembrane, and secreted proteins contains this domain. The prototype secreted-protein members of this family include short chain snake and frog toxins which in many cases bind with high-affinity to neuronal receptors and block their activity (5-7).

Until recently the only recognized secreted mammalian TFP/Ly-6/uPAR proteins were SLURP-1 (secreted mammalian Ly-6/uPAR-related protein) (8) and SLURP-2 (9) which are located on human chromosome 8q24.3 within a cluster of Ly-6-like human genes that otherwise code for GPI-linked proteins. Consistent with the putative ligand function of some secreted TFP/Ly-6/uPAR proteins, SLURP-1 was recently identified as a neuro-modulator of the α7 nicotinic receptor (ca nAChR), suggesting that it may regulate calcium homeostasis (10). It is clear that members of the secreted TFP/Ly-6/uPAR protein family, to which the PATE protein belongs, interact with partner proteins that, in many cases, are membrane-tethered receptors.

The human PATE gene is telomerically juxtaposed to the gene encoding acrosomal vesicle protein 1 (ACRV1), also known as the SP10 gene (11). Interestingly, the ACRV1 protein also contains 10 cysteine residues which conform to the TFP/Ly-6/uPAR domain, suggesting that the two genes ACRV1 and PATE may be part of a single chromosomal locus comprising TFP/Ly-6/uPAR genes.

The mouse Pate-B protein, (caltrin or sys7), modulates calcium permeability in sperm cells (17). Furthermore, PATE, ACRV1 and Pate B, all secreted TFP/Ly-6/uPAR domain-containing proteins, bind to specific sites on the sperm membrane (14, 16, 20).

Emes et al. investigated a syntenic region of rat (chromosome 8) mouse (chromosome 9) and human (chromosome 11) genomes suggesting that this region contains genes encoding Ly 6 homologous urinary proteins in all three species, and proposed that secreted urine Ly-6-like proteins function as proteinaceous pheromones in rodents (21).

Previously disclosed UniProt database accession numbers Q6UY27 (Clark et al) and AK123042 (Ota et al.) provide amino and nucleic acid sequences homologous to two of the PATE-like proteins of the invention, PATE M and PATE B respectively (under a different designation). However, no indication as to the tissue expression pattern, alternative splice forms and function of these sequences is provided.

### SUMMARY OF THE INVENTION

The present invention is based on the identification of novel human and mouse genes which code for secreted, cysteine-rich proteins expressed and hormonally regulated mainly in reproductive tissues and in the brain. Due to their unique genomic location and mode of tissue expression these genes were termed PATE-like genes.

The PATE-like genes described herein generate several splice isoforms which derive from exon skipping. Therefore, the nucleic acid molecules of the invention include various splice-variants resulting in nucleic acid molecules of differing sizes.

Accordingly, by a first of its aspects, the present invention provides an isolated polynucleotide encoding for a PATE-like protein comprising a sequence selected from the group consisting of:
SEQ ID No. 1 (encoding for PATE-DJ),
SEQ ID No. 5 (encoding for PATE-B exons 1 and 3),
SEQ ID No. 4 (encoding for PATE-M exons 1, 2 and 3),
SEQ ID No. 6 (encoding for PATE-M exons 1, 1a and 3),
SEQ ID No. 7 (encoding for PATE-M exons 1 and 3),
   and a polynucleotide having a degree of identity of at least about 70%, preferably at least about 80%, more preferably at least about 85%, also more preferably at least about 90%, and most preferably at least about 95%.

In another aspect, the present invention provides an isolated PATE like polypeptide comprising an amino acid sequence selected from the group consisting of:
SEQ ID No. 8 (PATE-DJ),
SEQ ID No. 12 (PATE-B “1, 3”, namely encoded by exons 1 and 3),
SEQ ID No. 11 (PATE-M “1, 2, 3”),
SEQ ID No. 13 (PATE-M “1, 1a, 3”),
SEQ ID No. 14 (PATE-M “1, 3”),
   and a polypeptide having a degree of identity of at least about 70%, preferably at least about 80%, more preferably at least about 85%, also more preferably at least about 90%, and most preferably at least about 95%.

The various polypeptides are termed according to the splice variant from which they were translated, i.e. PATE-B “1, 3” is encoded by exons 1 and 3 of the PATE-B gene, PATE-M “1, 2, 3” is encoded by exons 1, 2 and 3 of the PATE-M gene, PATE-M “1, 1a; 3” is encoded by exons 1, 1a and 3 of the PATE-M gene, and PATE-M “1, 3” is encoded by exons 1, and 3 of the PATE-M gene.

The short PATE-like polypeptide variants of the invention which lack the portion encoded by exon 2 comprise 5 cysteine residues. Such short variants are capable of forming multimeric protein aggregates conjugated via the “free”, unpaired cysteine residue.

Accordingly, in another aspect, the present invention provides an isolated multimeric polypeptide comprising at least two short variant PATE-like polypeptides conjugated via cysteine-cysteine bonds, wherein said short variant PATE-like polypeptides are selected from the group consisting of SEQ ID No. 12, SEQ ID No. 13, SEQ ID No. 14 and a short variant PATE-like polypeptide having a degree of identity of at least about 70%, preferably at least about 80%, more preferably at least about 85%, also more preferably at least about 90%, and most preferably at least about 95% to SEQ ID No. 12, 13 or 14, and wherein said short variant PATE-like polypeptides having 5 cysteine residues.

In various embodiments, the isolated multimeric polypeptide is a homodimer or a heterodimer comprising two short variant PATE-like polypeptides conjugated via cysteine-cysteine bonds. In one embodiment, the isolated multimeric polypeptide is a homodimer comprising two PATE B short variant polypeptides of SEQ ID NO. 12. In one embodiment the isolated multimeric polypeptide is a homodimer comprising two PATE M short variant polypeptides of SEQ ID NO 13 or SEQ ID NO 14. In one embodiment, the isolated multimeric polypeptide is a heterodimer comprising one PATE B short variant polypeptide of SEQ ID NO 12, and one PATE M short variant polypeptide of SEQ ID NO 13, or SEQ ID NO 14.

In another embodiment, the present invention provides a multimeric polypeptide wherein at least one of said short variant PATE-like polypeptides is an ACRV1ₛₘₐₗₗ polypeptide, namely a short variant of ACRV1 comprising only 5 cysteins.

In another aspect, the present invention provides a method of treating a disease or disorder comprising administering to a patient in need thereof a therapeutically effective amount of a pharmaceutical composition comprising a compound selected from the group consisting of
a. A molecule that interacts with a PATE-like polypeptide;
b. An antibody capable of specifically binding to an epitope of a PATE-like polypeptide;
c. A PATE-like polypeptide;
d. An agent that affects the synthesis or the secretion of PATE-like polypeptides from cells;
   wherein said PATE-like polypeptide being in accordance with any of the polypeptides or multimeric polypeptides of the invention provided above.

In one embodiment said disease or disorder are associated with the reproductive system. In one embodiment said disease or disorder are associated with prostate or testis.

In another embodiment, said disease or disorder is associated with body energy homeostasis, appetite, or food intake.

In another embodiment, said disease or disorder is associated with the central nervous system. In one embodiment, said disease or disorder is associated with nicotinic acetylcholine receptors. In one specific embodiment, said disease is Alzheimer's disease.

In one embodiment, the present invention provides a method of modulating nicotinic acetylcholine receptors (nAChR) comprising administering a therapeutically effective amount of at least one isolated PATE-like polypeptide in accordance with the invention to cells expressing said nAChR. In one embodiment, the present invention provides a method of increasing the net charge of α7 nAChR by administration of at least one isolated PATE-like polypeptide of the invention to cells expressing said nAChR. In one embodiment said PATE-like polypeptide is administered in a concentration of between about 10 nM and about 300 nM. In another embodiment said PATE-like polypeptide is administered in a concentration of between about 50 nM and about 250 nM. In one specific embodiment, said PATE-like polypeptide is hPATE-B.

In one embodiment, said method of modulating nicotinic acetylcholine receptors is performed in vitro.

In another aspect, the present invention provides a pharmaceutical composition comprising as an active ingredient a compound selected from the group consisting of
a. A molecule that interacts with a PATE-like polypeptide;
b. An antibody capable of specifically binding to an epitope of a PATE-like polypeptide;
c. A PATE-like polypeptide;
d. An agent that affects the synthesis or the secretion of PATE-like polypeptides from cells;
   and a pharmaceutically acceptable carrier; wherein said PATE-like polypeptide being in accordance with any of the polypeptides or multimeric polypeptides provided above.

In one embodiment said pharmaceutical composition is used for the treatment of reproductive-system related conditions.

In one embodiment said pharmaceutical composition is used for the treatment of body energy homeostasis related conditions. In one specific embodiment said pharmaceutical composition is used for treating obesity.

In one embodiment said pharmaceutical composition is used for treating disorders of the nervous system. In one specific embodiment said pharmaceutical composition is used for the treatment of neural-transmission related conditions. In one embodiment, said disorder is associated with nicotinic acetylcholine receptors. In one specific embodiment said pharmaceutical composition is used for treating Alzheimer's disease.

In another aspect, the present invention provides a method for diagnosing a nervous system disease or disorder comprising:
a. Obtaining a sample of nervous system tissue;
b. Preparing mRNA from said sample; and
c. Assessing the expression level of PATE-like mRNA in said sample;
   Wherein a decreased level of mRNA expression compared with normal nervous system tissue is indicative of a nervous system associated disease.

In one specific embodiment, said nervous system tissue is brain tissue. In one specific embodiment said disease or disorder is Alzheimer's disease.

In another aspect, the present invention provides a method for diagnosing a disease or disorder comprising
a. Obtaining a sample of a subject's body fluid;
b. Contacting said sample of body fluid with an antibody capable of specifically binding to an epitope of a PATE-like polypeptide;
c. Assessing the level of said PATE-like polypeptide in said sample;
wherein said PATE-like polypeptide being selected from the group consisting of SEQ. ID No. 8, 12, 13 and 14; and wherein a decreased or increased level of said PATE-like polypeptides compared to the level in normal subjects is indicative of a disease or disorder.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 is a schematic representation of the arrangement of genes within the locus comprising the human PATE-like genes and the corresponding syntenic murine genomic locus. FIG. 1A. The known human genes lie within a genomic segment on chromosome 11q24 initiating at the centromeric side with PKNOX2 at nucleotide 124,726,419 (numbering as in the Genome Browser May 2004 release), and terminating at the telomeric side with CDON at nucleotide 125,438,397. Arrows indicate direction of transcription. The genes coding for proteins containing the distinctive ten-cysteine motif are shown in red, and inactive pseudogenes are stippled. Accession numbers are ACRV1: NM_{—}001612, PATE: NM_{—}138294, PATE-M: NM 212555, PATE-B: AK123042. Accession number for PATE-DJ is pending. FIG. 1B. The syntenic murine (chromosome 9qA4) genomic locus. Note the 0.8 Mbp insertion in the mouse genome between Acrv1 and Pate-A. Accession numbers are Acrv1: NM_{—}007391, Pate-H: BY721155, Pate-Q: BQ032923, Pate-F: BY721010, Pate-A: AK020329, Pate-C: NM_{—}026593, Pate-E: AV379335, Pate: AK033745, Pate-M: BY721028, Pate-B (sys7): NM_{—}020264. Accession numbers for Pate-G, Pate-P, Pate-N and Pate-DJ are pending.

FIG. 2 is a photograph of a gel demonstrating differential expression of human PATE-like genes and flanking genes in various tissues. RT-PCR analysis of the human PATE-like genes and flanking genes was performed with cDNAs obtained from the indicated human tissues. Forward and reverse primers were chosen such that they always spanned an intron and the observed RT-PCR product at all times corresponded to the size expected of a spliced mRNA. For the ACRV1, PATE, PATE-M, PATE-DJ and PATE-B genes, the forward and reverse primers were located in the first and third exons (coding for the signal peptide and cysteines #6-#10, respectively). PCR was performed for 35 cycles [A] or 40 cycles [B]. Note that PIG8 (p53 induced gene 8) is ubiquitously expressed in all tissues, serving as a convenient internal control for cDNA integrity.

FIG. 3 is a schematic representation of the exon structure of PATE (Pate)-like genes. [A] The canonical exon structure of the PATE (Pate)-like genes is presented. Optional extra exons are represented as dotted boxes. The boxes represent the coding regions: signal peptide (SP); P1 containing C#1-C#5; P2 containing C#6-C#10N. The alternative splice forms comprise exons 1, (1a) and 3. [B] Demonstration of an alternative splice form comprising exons 1a and 3.

FIG. 4 is a photograph of a gel demonstrating Northern blot analyses of PATE B and PATE DJ expression. PATE B, PATE DJ and actin cDNAs were radioactively (³²P) labeled and each used to sequentially probe, under stringent wash conditions, a Northern blot (Clontech) of total RNA derived from the indicated human tissues. The blot was stripped between sequential probing. Expression of PATE B and PATE DJ mRNAs are clearly visible in prostatic and testicular tissues (lanes 8 and 9 respectively); no signal was seen in any other tissues examined.

FIG. 5 is a photograph of a gel demonstrating RT-PCR expression analyses of mouse Pate-like genes. RT-PCR analyses of the mouse Pate-like genes were performed with cDNAs (Clontech) obtained from different mouse tissues as indicated. Forward and reverse primers were chosen such that they always spanned an intron, and all RT-PCR products corresponded to the sizes expected of spliced mRNA. Results presented here used forward and reverse primers located in the second and third exons (coding for cysteines #1-#5 and cysteines #6-#10, respectively); similar results were obtained when the analysis was repeated with forward and reverse primers located in the first and third exons (data not shown). PCR was performed for 35 cycles and the PCR products analyzed as described in Methods. The ubiquitously expressed mouse G3PDH served as a control for cDNA integrity.

FIG. 6 is a photograph of a gel demonstrating the effect of castration and subsequent DHT administration on Pate-like gene expression in the dorsal and ventral lobes of the mouse prostate. Mice were castrated and fourteen days later injected (sc.) at time 0, 24 hours, and 48 hours either with oil or with dihydroxytestosterone (DHT) dissolved in the oil. Mice were sacrificed twelve minutes, twenty-four, forty-eight and seventy-two hours following these injections (0.2, 24, 48 and 72 respectively) and the ventral (VP) and dorsal (DP) prostate lobes isolated, followed by RNA isolation and cDNA preparation. RT-PCR analyses were. performed using forward and reverse primers located in the first and third exons. PCR was performed for 35 cycles and the PCR products analyzed as described in Methods. The ubiquitously expressed mouse L19 gene served as a control for cDNA integrity. [unx, uncastrated].

FIG. 7 is a photograph demonstrating Pate-like gene expression in the mouse virgin, pregnant and lactating mammary gland. Mammary gland tissue was isolated from virgin mice (V), mice at 4, 8, 12 and 16 days of pregnancy (P4, P8, P12 and P16, respectively) and from mice lactating for 1 and 7 days (L1 and L7, respectively), followed by RNA isolation and cDNA preparation. RT-PCR analyses were performed using forward and reverse primers located in the first and third exons. PCR was performed for 35 cycles and the PCR products analyzed as described in Methods. The ubiquitously expressed mouse L19. gene served as a control for cDNA integrity. The epithelial tissue of pregnant mammary glands display increased level of lateral branching, and noticeable alveoli in the pregnant mammary glands (days 8 and 16 of pregnancy, respectively) compared with (virgin, 13w) virgin glands.

FIG. 8 is a photograph of a gel demonstrating expression of Pate, Pate-P and Pate-C proteins-N-glycosylation of Pate and Pate-C in HK293 (human kidney) cells. [A] HK293 cells were transfected with constructs encoding the proteins that comprised the Pate, Pate-P or Pate-C sequences, tagged at their N-termini with a Flag epitope and the secreted Pate-like proteins purified as described in Methods. The purified proteins were analyzed by 11% SDS-PAGE, Western blotted and probed with anti-Flag antibodies [A and B]. [B] Western blot analysis of Flag-tagged Pate-like proteins treated with PNGase F. Proteins were treated as described in [A], with the difference that the proteins were treated with PNGase F to remove N-linked sugars (lanes 2 and 4 for Pate and Pate-C, respectively).

FIG. 9 is a multiple sequence alignment of C₁₀N motif and a schematic representation of a phylogenetic tree of PATE (Pate)-like proteins from human, mouse, rat and dog. [A] The amino acid sequences of the human, mouse, rat and dog PATE (Pate)-like proteins are presented. The first (P1) and second (P2) blocks show sequences extending from C#1 to C#5 and C#6 to C#10N respectively. The C₁₀N motifs are highlighted. Wherever possible, Pate-like nomenclature has been used for the interspecies orthologs. Otherwise arbitrary designations, such as RO5, were used for illustration. Expression of all human and mouse PATE (Pate)-like genes presented have been demonstrated experimentally by RT-PCR analyses. [B] The same multiple sequence alignment was used to build a phylogenetic tree. The tree is a Neighbor-Joining consensus tree based on 1000 replicates. Apparent orthologous groups are marked in gray background.

FIG. 10 is a photograph of a gel demonstrating sesame oil induction of Pate, Pate-E, and Pate-B gene expression in the ventral prostate. Mice were injected (sc.) at time 0 hours with sesame oil. Mice were sacrificed twelve minutes and twenty four hours following these injections (0.2, 24 respectively). mRNA was isolated from the ventral prostate lobe followed by cDNA preparation. RT-PCR analyses were performed using forward and reverse primers that spanned an intron and the observed RT-PCR product at all times corresponded to the size expected of a sliced mRNA. PCR was performed for the indicated number of cycles. Note that the Pate/H gene is expressed to a similar extent at the 0.2 and 24 hours time points serving as a convenient internal control for equal amounts of intact cDNA in the samples.

FIG. 11 is a photograph of a gel demonstrating RT-PCR expression analyses of human PATE-like genes in different regions of human brain tissue.

[A] RT-PCR analyses of the human PATE-like genes were performed with cDNAs (from Biochain Institute, Inc.) obtained from different regions of the human brain, as indicated. Forward and reverse primers were chosen such that they always spanned an intron, and all RT-PCR products corresponded to the sizes expected of spliced mRNA. Results presented here used forward and reverse primers located in the first and third exons (coding for the signal peptide and cysteines #6410, respectively). PCR was performed for 40 cycles and the PCR products analyzed by agarose gel electrophoresis.
[B] A semi-quantitative comparative analysis of PATE-M expression in testis and brain cerebral cortex.
[C] Expression of PATE-Mₛₘₐₗₗ isoform in two independent cerebral cortex samples (i and ii, isolated from two different individuals) and two independent temporal lobe samples (i and ii, isolated from two different individuals). As a control, PATE-M expression in testis (tes.) demonstrated as expected, expression of both PATE-M and PATE-Mₛₘₐₗₗ isoforms.

FIG. 12 is a photograph of a gel demonstrating decreased expression of PATE-M (exons 1, 1a and 3) in Alzheimer diseased brain. cDNAs prepared from different individual regions of normal (lanes 4, 5 and 6) or Alzheimer (lanes 1 and 2) brains were subjected to RT-PCR analyses. Lanes 1 and 2 represent reactions performed with temporal lobe cDNAs prepared from two different male Alzheimer patients aged 60 and 65 years, respectively. Analysis of the PATE-M gene (panel A) was performed with forward and reverse primers located in exons 1 and 3 of the PATE-M gene. Analysis of actin expression (panel B) was used to assure that equal amounts of cDNA were used. The dotted ellipses (panel A) demonstrate significantly lower PATE-M levels in the Alzheimer temporal brain samples (lanes 1 and 2) as compared to normal temporal lobe sample (lane 4). As a negative control for PATE-M expression cDNA from human liver (lane 3) was included—lane 7 represents analyses in which no cDNA was added to the reaction.

FIG. 13 is a graph demonstrating modulation of the activity of nicotinic acetylcholine receptors (nAChRs) by PATE (Pate)-like proteins.

FIG. 14 is a photograph of a gel demonstrating the generation of a monomer as well as a dimer of PATE-M “1a, 3”.

[A] Electrophoresis in the absence (−) of β-mercaptoethanol (β-MSH), i.e. under non-reducing conditions. [B] Electrophoresis in the presence (+) of β-mercaptoethanol (β-MSH), i.e. under reducing conditions. The left lane shows a molecular weight ladder indicating the size of the polypeptide in kDa.

Table 1. Sequences of the human PATE-like and mouse Pate-like proteins and signal peptide prediction. The amino acid sequences of the human PATE-like (and mouse Pate-like) proteins are presented starting with the initiating methionine. Upper Panel Sequences were subjected to SignalP analysis-signal peptide probabilities and the predicted signal peptide cleavage sites are presented. The upward facing arrow designates the predicted cleavage site of the signal peptide and the red dots indicate the exon boundaries. Hydrophobic amino acid residues downstream to the initiating methionine, and likely comprising the signal peptide, are highlighted. Lower Panel Exons 2 and 3, comprising cysteines #145 and cysteines #6-#10, respectively.

### DETAILED DESCRIPTION OF EXEMPLARY EMBODIMENTS

The present invention is based on the identification of novel human and mouse genes which code for secreted, cysteine-rich proteins expressed and hormonally regulated mainly in reproductive tissues. Due to their expression pattern, chromosomal localization and unique structure the genes have been denominated PATE-like genes. (In the context of the present invention the terms PATE-like or PATE- (in italics) like are used interchangeably). These genes include three human PATE-like genes (PATE-M, PATE-DJ and PATE-B) that co-localize with the ACRV1 and PATE genomic locus. These novel PATE-like genes code for secreted proteins containing the typical TFP/Ly-6/uPAR domain. Significantly, all show selective expression in male reproductive tissues, i.e. prostate and/or testis. This does not rule out the possibility that these human PATE-like genes will, under particular conditions, be expressed in female-specific reproductive tissues.

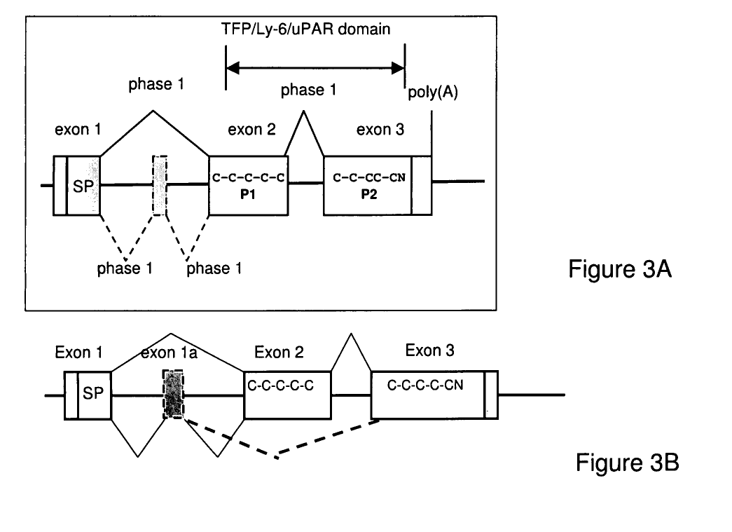

The human PATE-like nucleic acids of the invention code for proteins which comprise a putative N-terminal signal peptide and ten conserved cysteine residues. The N-terminal signal peptide is encoded by the first exon (exon 1), whereas protein domains containing cysteines #145 and cysteines #6410 are encoded by two separate 3′ exons (exons 2 and 3 respectively, shown in FIG. 3A). For the PATE-M gene, 1 additional exon, designated 1a is present between exons 1 and 2, and codes for a small number of amino acids.

PATE-B and PATE-M generate two splice isoforms which derive from exon 2 skipping. Both isoforms comprise the putative N-terminal signal peptide but whereas the larger transcript codes for proteins comprising all ten cysteine residues, the smaller transcript codes only for cysteines #6-#10.

Accordingly, the nucleic acid molecules of the invention include various splice-variants resulting in nucleic acid molecules of differing sizes. In a preferred embodiment the nucleic acids of the invention comprise the nucleic acid sequences denoted as SEQ ID Nos. 1-7 which encode for PATE DJ (SEQ ID No. 1: exons 1, 2 and 3), PATE B (SEQ ID No. 2: exons 1, 2 and 3), PATE M (SEQ ID No. 3: exons 1, 1a, 2 and 3), a transcript of PATE M excluding exon 1a (SEQ ID No. 4; exons 1, 2 and 3) a short transcript of PATE B comprising exons 1 and 3 (SEQ ID No. 5), a short transcript of PATE M comprising exons 1, 1a and 3 (SEQ ID No. 6) and another short transcript of PATE M comprising only exons 1 and 3 (SEQ ID No. 7). The invention also concerns homologues of these nucleic acids having at least about 70%, preferably at least about 80%, more preferably at least about 85%, also more preferably at least about 90%, and most preferably at least about 95% homology thereto.

In another aspect the present invention concerns PATE-like polypeptides, preferably PATE-like polypeptides comprising an amino acid sequence as denoted in SEQ ID Nos. 8-14 which encode for PATE DJ (SEQ ID No. 8), PATE B (SEQ ED No. 9), PATE M (SEQ ID No. 10), PATE M excluding exon 1a (SEQ ID No. 11), a short polypeptide of PATE B translated from exons 1 and 3 (SEQ ID No. 12) a short polypeptide of PATE M translated from exons 1, 1a and 3 (SEQ ID No. 13) and another short polypeptide of PATE M translated from exons 1 and 3 (SEQ ID No. 14). The invention also concerns homologues of these polypeptides having at least about 70%, preferably at least about 80%, more preferably at least about 85%, also more preferably at least about 90%, and most preferably at least about 95% homology thereto.

In accordance with the present invention, amino acid sequence homology is measured in percentage. Homologues of PATE-like polypeptides of the present invention will possess a relatively high degree of sequence identity upon alignment using standard techniques and commercially available software packages for sequence alignment and comparison, the methods of which are well known in the art (e.g. Altschul, et al., Nature Genet., 6: 119, 1994).

The Basic Local Alignment Search Tool, for example, also known as BLAST, can be found in various sources such as the National Center for Biotechnology Information (NCBI). It is also available for on-line use. BLAST utilizes numerous sequence analysis variant programs such as blastp, blastn, blastx, tblastn and tblastx. The short PATE-like polypeptide variants having only 5 cysteine residues are capable of forming multimeric protein aggregates conjugated via the “free”, unpaired cysteine residue. Such multimeric proteins may be homogeneous and comprise a single type of PATE-like protein e.g. PATE B or PATE M, or may be heterogeneous and comprise a mixture of PATE B and PATE M short variant proteins. Preferably, the multimeric protein comprises two short PATE-like protein variants and is a homodimer of PATE B or PATE M, or a heterodimer comprising one PATE B and one PATE M short variant.

In addition, due to differential splicing ACRV1 may also be expressed as a “small” polypeptide comprising only 5 cysteine residues (for example, UniProt database accession number NP_{—}064500) and may be conjugated with the above described small Pate-like polypeptide variants to generate protein aggregates.

The following are non-limiting examples of disulfide-linked, homo and hetero dimers of the PATE-like polypeptides of the invention:

PATE-Mₛₘₐₗₗ with exon 1a [5 cysteines]+PATE-Mₛₘₐₗₗ with exon 1a [5 cysteines]
PATE-Mₛₘₐₗₗ without exon 1a [5 cysteines]+PATE-Kₛₘₐₗₗ with exon 1a [5 cysteines]
PATE-Mₛₘₐₗₗ without exon 1a [5 cysteines]+PATE-Mₛₘₐₗₗ without exon 1a [5 cysteines]
PATE-Bₛₘₐₗₗ [5 cysteines]+PATE-Mₛₘₐₗₗ with exon 1a [5 cysteines]
PATE-Bₛₘₐₗₗ [5 cysteines]+PATE-Mₛₘₐₗₗ without exon 1a [5 cysteines]
PATE-Bₛₘₐₗₗ [5 cysteines]+PATE-Bₛₘₐₗₗ [5 cysteines]
ACRV1ₛₘₐₗₗ [5 cysteines]+ACRV1ₛₘₐₗₗ [5 cysteines]
ACRV1ₛₘₐₗₗ [5 cysteines]+PATE-Mₛₘₐₗₗ with exon 1a [5 cysteines]
ACRV1ₛₘₐₗₗ [5 cysteines]+PATE-Mₛₘₐₗₗ without exon 1a [5 cysteines]
ACRV1ₛₘₐₗₗ [5 cysteines]+PATE-Bₛₘₐₗₗ [5 cysteines]

The PATE-like polypeptides of the invention may also be modified using methods well known in the art. Such modifications include but are not limited to glycosylation and conjugation to additional molecules e.g. polypeptides.

Therefore, in a further aspect, the invention concerns a polypeptide PATE-like conjugate, which comprises an amino acid sequence of a PATE-like polypeptide or homologues of these polypeptides having at least about 70%, preferably at least about 80%, more preferably at least about 85%, also more preferably at least about 90%, and most preferably at least about 95% homology thereto, further comprising an attachment group for a non-polypeptide moiety.

For the purposes of the present invention the term “conjugate” is intended to indicate a molecule formed by the covalent attachment of one or more polypeptides to one or more non-polypeptide moieties such as lipophilic compounds, carbohydrate moieties, or oligosaccharide moiety. By way of non-limiting example, the non-peptide moiety is an oligosaccharide moiety and the conjugation is to be achieved by N-glycosylation.

The term “attachment group” used herein is intended to indicate an amino acid residue group of the polypeptide capable of coupling to the relevant non-polypeptide moiety.

Modification of the PATE-like polypeptides of the present invention includes N-glycosylation site altered in such a manner that either a functional N-glycosylation site is introduced into the amino acid sequence or removed from said sequence.

By either removing or introducing an amino acid residue from the PATE-like polypeptides it is possible to specifically modulate the polypeptide so as to make the molecule more susceptible to conjugation to the non-polypeptide moiety of choice as described above, or to modulate PATE-like polypeptides activity.

The term “amino acid residue” is primarily intended to indicate an amino acid residue contained in the group consisting of the 20 naturally occurring amino acids: i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or 1), lysine (Lys or K), leucine (Leu or L), methionine (Met or M), asparagine (Mn or N), proline (Pro or P), glutamine (Gin or Q), arginine (Arg or R), serine (Ser or S), threonine (Thr or T), valine (Val or V), tryptophan (Trp or W), and tyrosine (Tyr or Y) residues. In addition, the orthologous mouse Pate, Pate-M, Pate-DJ and Pate-B genes were also identified. These genes localize centromerically to the mouse Acrv1/sp10 gene. Remarkably, the mouse Pate-like genomic locus comprises an additional nine transcriptionally-active Pate-like genes which all encode secreted TFP/Ly-6/uPAR-domain-containing proteins, while in the human genome these mouse Pate-like genes are either inactive (two genes) or completely absent (the remaining seven genes). These mouse Pate-like genes are selectively expressed in prostate, testis, brain, placenta, the pregnant and lactating mouse mammary gland. In addition, specific effects of castration and subsequent testosterone administration on their expression in prostate, all indicate that these genes function in both male and female-related reproductive activities and are likely hormonally regulated.

The PATE-like polypeptides of the invention are, therefore, contemplated to be useful as such for therapeutic, diagnostic or other purposes as disclosed herein.

The PATE-like polypeptides of the present invention can be of any species, and in particular, of mammalian origin (e.g. mouse Pate-like polypeptides), more particularly of human origin.

In one of its aspects, the present invention provides a method of treatment of a disease, condition or disorder comprising administering to a patient in need thereof a therapeutically effective amount of a pharmaceutical composition comprising a compound selected from the group consisting of
a. A molecule that interacts with a PATE-like polypeptide;
b. An antibody capable of specifically binding to an epitope of a PATE-like polypeptide;
c. A PATE-like polypeptide;
d. An agent that affects the synthesis or the secretion of PATE-like polypeptides from cells;

We hereby propose that the secreted polypeptides of the invention can be used to modulate reproductive-system associated processes.

In addition, the inventors provide a surprising indication that PATE-like genes are involved in energy homeostasis and response to food intake. Accordingly, the secreted polypeptides of the invention can also be used to modulate diseases or disorders that are associated with energy homeostasis, appetite or food intake, e.g. obesity.

Furthermore, because of the extensive expression of a particular alternative splice form of PATE-M in brain tissue and of PATE-B in spinal cord tissue, the PATE-like genes appear to be connected to neural-related functions, and the secreted polypeptides of the invention can therefore be used to modulate diseases or disorders that are associated with the central nervous system.

Specifically, as shown in the Example 4 below, a decreased expression of a PATE-like polypeptide (PATE-M) was demonstrated in the brains of Alzheimer disease patients. Furthermore, it has been experimentally shown (see example 5 below) that PATE-like polypeptides modulate the activity of acetylcholine receptor. Therefore, in one specific embodiment, the present invention provides use of the PATE-like polypeptides of the invention for the treatment of Alzheimer's disease, or use of the PATE-like polypeptides for the manufacture of a pharmaceutical composition for the treatment of Alzheimer's disease.

Alzheimer's disease (AD) is characterized by the gradual degeneration of cholinergic neurons and accumulation of β-amyloid peptides. Recently, it has been demonstrated that there is a crucial impairment of nicotinic acetylcholine receptor (nAChR) binding cites in the brain of AD patients (for review see reference 24). As a result nAChR has been suggested to play a role in mediating both β-amyloid toxicity and neural degeneration and to serve as a therapeutic target for the treatment of AD. The present invention demonstrates for the first time the effect of PATE-like polypeptides on the activity of nAChR, and thus the potential role of PATE-like polypeptides in the treatment of AD.

Diseases or disorders which are characterized by a decreased level of PATE-like polypeptides can be treated by administering to a patient in need thereof the PATE-like polypeptides of the invention.

On the other hand, diseases or disorders that are associated with over expression of PATE-like genes can be treated by eliminating or reducing the amount of PATE-like polypeptides, e.g. using antibodies, or siRNA directed to the PATE-like polynucleotides.

Another aspect of the invention concerns a method of diagnosing a disease associated with an altered level of PATE-like polypeptides using antibodies directed against an epitope of the PATE-like polypeptides of the invention. The expression “altered level” denotes either a high or a low level of the polypeptides.

In a further aspect, the present invention discloses a composition comprising a PATE-like polypeptide or PATE-like polypeptide conjugate and at least one pharmaceutically acceptable carrier or excipient.

The PATE-like polypeptide or PATE-like polypeptide conjugate, or the pharmaceutical composition according to the invention may be used for the treatment of diseases or disorders that are associated with over expression of PATE-like genes, and diseases or disorders which are characterized by a decreased level of PATE-like polypeptides.

The PATE-like polypeptide or PATE-like polypeptide conjugate, or the pharmaceutical composition according to the invention may be further used for the treatment of diseases or disorders of the reproductive-system associated processes, and in particular, to modulate reproductive-system associated processes.

In yet another aspect, the PATE-like polypeptide or PATE-like polypeptide conjugate, or the pharmaceutical composition according to the invention may be further used for the treatment of diseases or disorders that are associated with energy homeostasis, appetite or food intake, such as, but not limited to obesity.

In another aspect, the PATE-like polypeptide or PATE-like polypeptide conjugate, or the pharmaceutical composition according to the invention may be further used for the treatment of neural-related functions, and the secreted polypeptides of the invention may therefore be used to modulate diseases or disorders that are associated with the central nervous system.

The PATE-like polypeptide or PATE-like polypeptide conjugate of the invention will be administered to patients in a therapeutically effective amount or dosage.

In the present invention, “therapeutically effective amount” shall mean a dose that is sufficient to produce the desired effects in relation to the condition treated. The specific dose regimen will depend on the particular disease and/or disorder which are treated, and will be ascertainable by one skilled in the art using known techniques.

A suitable dose of PATE-like polypeptides or PATE-like polypeptide conjugate of the invention is contemplated to be in the range of about 2-500 microgram/kg body weight, such as in the range of 5-400 microgram/kg, or in the range such as 15-300 microgram/kg.

A person skilled in the art will appreciate that an effective amount of a PATE-like polypeptide or PATE-like polypeptide conjugate of the invention depends upon the particular disease or disorder being treated, the dose together with the administration regimen. A person skilled in the art will take in account whether a PATE-like polypeptide or PATE-like polypeptide conjugate is administered alone or in conjunction with other drugs or other pharmaceutical agents, and the health of the patient treated. A person skilled in the art will also consider whether a PATE-like polypeptide or PATE-like polypeptide conjugate is administered systemically or locally.

Typically, a PATE-like polypeptide, PATE-like polypeptide conjugate or an antagonist of a PATE-like polypeptide (e.g. antibody) is administered in an effective dose sufficient to normalize expression of PATE-like polypeptides in the patient being treated. Normalization may be determined by whether the said patient exhibits over expression, reduced expression in comparison to standard level of expression typical for a person of the patient age, sex and other characteristics.

For the purpose of the present invention, the term “patient” shall mean mammals, specifically humans.

The PATE-like polypeptides or PATE-like polypeptide conjugates of the present invention are administered in a composition comprising one or more pharmaceutically acceptable carriers or excipients.

The PATE-like polypeptides or PATE-like polypeptide conjugates of the present invention can be formulated to produce pharmaceutical compositions in a manner known in the art to achieve sufficient storage stability and suitability for administration to humans or other mammals.

Said pharmaceutical compositions can be designed in a variety of forms, including: liquid, gel, lyophilized form, or any other suitable form know in the art. The particular form is selected according to the disease, disorder, or condition being treated and will be understood to a person of skill in the art.

Where the pharmaceutical compositions are prepared in lyophilized form addition of one or more pharmaceutically acceptable diluents is required prior to consumption. A non-limiting example of diluents is sterile water or sterile physiological saline solution.

The PATE-like polypeptides or PATE-like polypeptide conjugates of the present invention can be used in a salt form thereof or in another form. Several salts can be used for that purpose, such as but not limited to, salts with alkali metals, sodium, potassium, calcium, or magnesium. Said salts or complexes thereof can have a crystalline structure or an amorphous structure.

Pharmaceutically acceptable carrier or excipient shall mean a carrier or excipient which does not cause any undesired effects in the patients treated taking into account the amounts and concentrations of the PATE-like polypeptides or PATE-like polypeptide conjugates administered. The specific selection and utilization of pharmaceutically acceptable carriers and excipients are well known in the art.

The pharmaceutical compositions of the invention may be administered alone or in combination together with other pharmaceutical agents. The pharmaceutical compositions of the invention can therefore incorporate other pharmaceutical agents or they can be administered apart from the PATE-like polypeptides or PATE-like polypeptide conjugates, either simultaneously or in accordance with another treatment regimen.

In another aspect, the PATE-like polypeptides or PATE-like polypeptide conjugates of the present invention can be used as an adjuvant or a synergist to other therapies.

The administration of the pharmaceutical compositions of the present invention is not limited to a particular route. The pharmaceutical compositions of the present invention can therefore be administered subcutaneously, intravenously, intraperitoneally, intramuscularly, orally, intracerebrally, intrapulmonary, intranasally, transdermally, vaginally, rectally, intraocularly. Said compositions can be further administered in any other manner acceptable by the man skilled in the art.

The pharmaceutical compositions of the present invention can be administered by infusion, or by injection and other techniques known in the art.

The pharmaceutical compositions of the present invention may optionally comprise surfactants or detergents for the purpose of solubilization of the active ingredient as well as to protect the PATE-like polypeptides or PATE-like polypeptide conjugates against aggregation, or denaturation of said polypeptides. Suitable surfactants are known to the person skilled in the art.

The PATE-like polypeptides or PATE-like polypeptide conjugates may also be encapsulated in microcapsules prepared, for example, by interfacial polymerization. The PATE-like polypeptides or PATE-like polypeptides conjugates may also be delivered utilizing other drug delivery systems such as but not limited to liposomes.

In a further embodiment, the present invention relates to an antibody that binds specifically to PATE-like polypeptides or PATE-like polypeptide conjugates of the present invention or to a specific fragment or epitope of said polypeptides.

The antibodies of the present invention can be used to identify, bind to or neutralize PATE-like polypeptides or PATE-like polypeptide conjugates in any organism.

These antibodies can be monoclonal antibodies, polyclonal antibodies or synthetic antibodies as well as fragments of antibodies. Monoclonal antibodies can be prepared, in variety of techniques known in the art, such as, but not limited to fusion of mouse myeloma cells to spleen cells derived from immunized mammals. These antibodies of the present invention can be used, by way of non-limiting example, for the immunoprecipitation and immunolocalization of PATE-like polypeptides or PATE-like polypeptide conjugates according to the invention.

### EXPERIMENTAL PROCEDURES

Materials and antibodies—Unless otherwise specified, chemicals and reagents were obtained from Sigma (St. Louis, Mo.). The anti-Flag antibodies were affinity-purified rabbit polyclonal antibodies.

### RT-PCR Analyses of the Human and Mouse PATE (Pate)-Like Genes

Forward and reverse oligonucleotide primers were synthesized using the DNA sequences obtained from the PATE (Pate)-like sequences (see below Bioinformatic strategies). RT-PCR analysis of the human PATE-like genes (and flanking genes) as well as the mouse Pate-like genes was performed with cDNAs obtained from different human or mouse tissues (Clontech) as indicated. Forward and reverse primers were chosen such that they always spanned an intron and the observed RT-PCR product at all times corresponded to the size expected of a spliced mRNA.

Sequencing of PATE (Pate)-Like cDNAs

All human PATE-like cDNAs were either directly sequenced from gel purified RT-PCR DNAs or alternatively the gel-purified cDNAs were cloned into TOPO4 (InVitrogen) plasmids and then sequenced. For the mouse Pate-like cDNAs, RT-PCR generated DNAs were gel purified and directly sequenced.

### Bioinformatic Strategies for Identification of PATE (Pate)-Like Genes

The sequence homology among the TFP family of proteins is generally low except for the common pattern of cysteines in the sequence and an initial attempt to identify PATE-like genes by using conventional protein homolog search programs such as BLAST or BLAT was not successful. However, a careful inspection of protein sequences and exon structures of initially identified PATE and PATE-like genes enabled us to devise a method to detect PATE-like genes in the genomic sequences. We developed two protein sequence patterns, P1 and P2, to represent C#1-C#5 and C#6-C#10N patterns, respectively, found in PATE-like proteins. The two patterns are “C-X(2)-C-X(5,10)-C-X(3,8)-C-X(4,9)-C” for P1 and “C-X(2,4)-C-X(11,20)-C-C-X(2,7)-C-N” for P2, where X(n,m) denotes a stretch of any amino acids ranging in length from n to in. The patterns were reverse-translated and transformed to Perl regular expressions. We searched the genomic locus bounded by PKNOX2 and CDON genes in the human genome (May 2004 freeze) and orthologous loci in the mouse genome (May 2004 freeze), in the rat genome (June 2003 freeze) and in the dog genome (July 2004 freeze). Two exons bearing P1 and P2, respectively, were separately searched. Then, closely (less than 10 kb) located P1 and P2 pairs were joined to form single genes. In human and mouse, the precise exon boundaries were predicted by manually inspecting all possible intron-exon boundaries for those that will maintain the open reading frame and the experimentally determined protein sequence. The corresponding signal peptide-bearing exon was located in the 5′ flanking region of each gene. The putative gene structure was verified by expressed sequence tag search, by comparing each gene with a respective ortholog in human or mouse, or experimentally by RT-PCR, cloning and sequencing. Possible orthologous relationships among PATE-like genes from human, mouse, rat and dog were inferred by a phylogenetic analysis using MEGA3 program (12) based on a multiple sequence alignment of P1 and P2 sequences obtained by using T-Coffee program (13).

### Generation of Eucaryotic Expression Constructs and Fusion Proteins

Cloning was conducted with the eucaryotic expression vector pCMV3 (Sigma) via selected restriction sites. This vector codes for the preprotrypsin signal peptide followed by sequences coding for the Flag epitope. DNA coding for the human Fc fragment (hFc) was inserted 3′ to the Flag epitope. A cleavage site for the highly specific TEV (tomato etch virus) protease was also introduced between the C-terminal of the Pate-like proteins and the hFc segment. cDNA fragments encoding the Pate-like proteins were subcloned in-frame into the pCMV3 (5′FlaghFc3′) vector to render pCMV3 as 5′Flag-Pate-like-TEV-hFc3′.

### Generation of HK293 Transfectants Expressing Flag-Pate-TEV-hFc Proteins

HK293 (human kidney) cells were transiently transfected with the eucaryotic pCMV3 expression vectors (6 μg DNA/25 cm² flask) coding for the Flag-(Pate-like)-TEV-hFc fusion proteins. The secreted C-terminally hFc tagged Flag-Pate-like-TEV-hFc proteins contained in the conditioned media (CM) were collected on Protein A-Sepharose 4 Fast Flow resin (Amersham Pharmacia Biotech) and the N-terminal Pate-like proteins were released by incubating the Protein A beads with TEV protease.

### SDS-Polyacrylamide Protein Gel Electrophoresis (SDS-PAGE)

SDS-polyacrylamide gel for protein separation was performed as previously described, and blots were reacted with polyclonal rabbit anti-Flag primary antibody. For glycosidase treatment, Protein A purified proteins were incubated with 1U PNGase F (NEB).

Expression in Xenopus oocytes

Mature (>9 cm) female Xenopus laevis African toads (Nasco, Ft. Atkinson, Wis.) were used as a source of oocytes. Prior to surgery, frogs were anesthetized by placing the animal in a 1.5 g/L solution of MS222 (3-aminobenzoic acid ethyl ester; Sigma) for 30 min. Oocytes were removed from an incision made in the abdomen.

In order to remove the follicular cell layer, harvested oocytes were treated with 1.25 mg/ml collagenase (Worthington Biochemical Corporation, Freehold, N.J.) for 2 hours at room temperature in calcium-free Barth's solution (88 mM NaCl, 1 mM KC1, 2.38 mM NaHCO₃, 0.82 mM MgSO₄, 15 mM HEPES (pH 7.6), 0.1 mg/ml gentamicin sulfate). Subsequently, stage 5 oocytes were isolated and injected with 50 nl (5-20 ng) each of the appropriate subunit cRNAs. Recordings were made 3 to 5 days after injection.

### Electrophysiology

Experiments were conducted using OpusXpress 6000A (Axon Instruments, Union City Calif.). OpusXpress is an integrated system that provides automated impalement and voltage clamp of up to eight oocytes in parallel. Cells were automatically perfused with bath solution, and agonist solutions were delivered from a 96-well plate. Both the voltage and current electrodes were filled with 3 M KCl. The agonist solutions were applied via disposable tips, which eliminated any possibility of cross-contamination. Cells were voltage-clamped at a holding potential of −60 mV. Data were collected at 50 Hz and filtered at 20 Hz. ACh applications were 8 seconds with 241 second wash periods. Each oocyte received two initial control applications of ACh, then the PATE-like peptides were pre-applied for 241 seconds at the indicated concentrations through an alternative supply of bath solution. Subsequently the PATES were co-applied with ACh at the control concentration. The control ACh concentrations for α7 and α4β2, receptors were 60 μM and 30 μM, respectively. Responses to the ACh PATE co-application were calculated relative to the preceding ACh control responses based on net charge. Net charge was integrated for the entire response, i.e. until the currents return to baseline, such that the total time window for the net charge measurement was 120 seconds, beginning 2 seconds prior to the ACh delivery. Responses of at least four oocytes were measured for each experimental concentration. Statistical analyses of PATE effects were based on pairwise T-test between the responses of each oocyte to ACh alone or ACh plus PATE peptide, following pre-incubation with the PATEs.

### Results

### Example 1

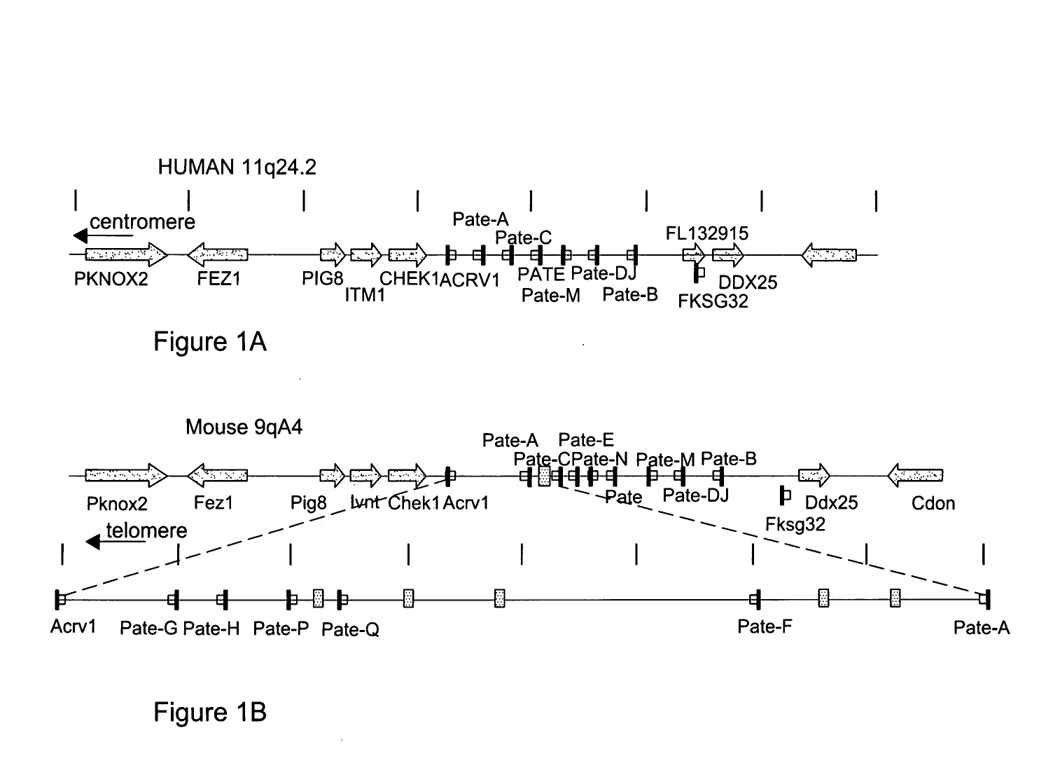

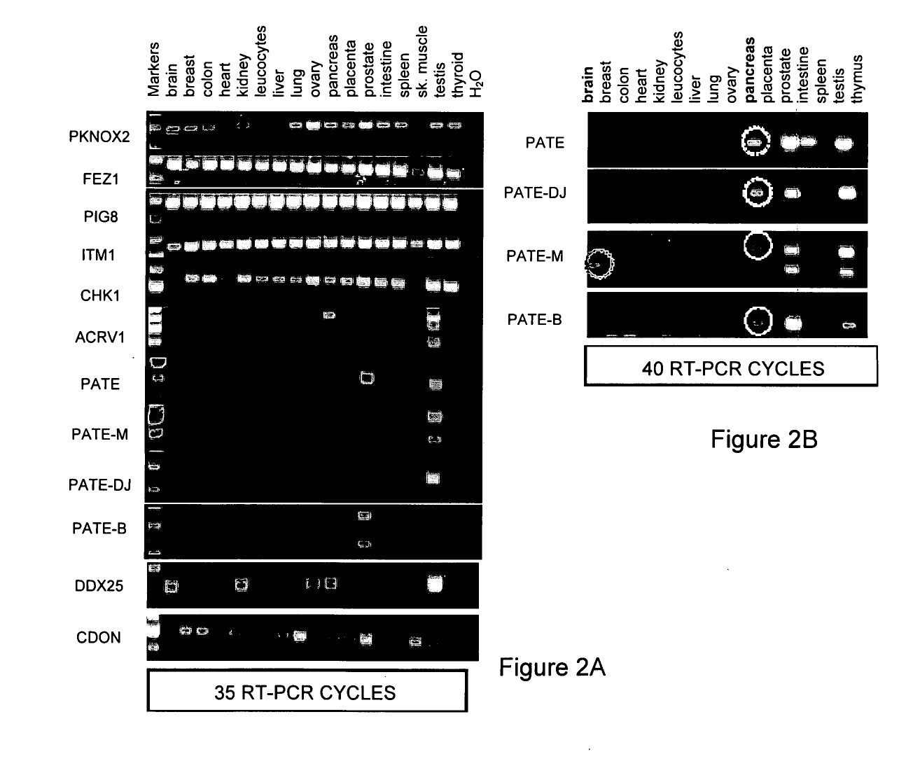

Identification of a human PATE-like gene cluster The PATE gene codes for a small, cysteine-rich protein, selectively expressed in human male reproductive tissues including prostate, testis, epididymis and seminal vesicle (1, 14). Pattern-search techniques (see the Methods section) revealed three additional PATE-like genes, designated PATE-B, PATE-M and PATE-DJ which localized to the same 11q24 genomic locus as the PATE gene (FIG. 1). Expression analyses of these genes by RT-PCR in seventeen different human tissues demonstrated selective expression in prostatic or/and testicular tissue with negligible expression in all other tissues (FIG. 2). The PATE-B gene was expressed primarily in prostate with lesser expression in testis whereas the PATE-M and PATE-DJ genes showed a reverse pattern of expression (FIG. 2). RT-PCR analyses of all known genes within a stretch of 700 kbp comprising the human PATE-like genes, demonstrated that the non-PATE-like genes did not show any preferential expression in reproductive tissues (FIG. 2). Consistent with previously published data, ACRV1 (acrosomal vesicle protein 1 gene also known as SP10) demonstrated multiple splice isoforms, expressed primarily in testicular tissue (11), and also in pancreatic tissue (FIG. 2)

Sequencing full-length cDNAs showed that the human PATE-like genes code for similar proteins that all comprise a putative N-terminal signal peptide (see below) and ten conserved cysteine residues (Table 1). Comparison of genomic and cDNA sequences showed that in all PATE-like proteins the N-terminal signal peptide is encoded by the first exon (exon 1), whereas protein domains containing cysteines #1-#5 and cysteines #6-410 are encoded by two separate 3′ exons (exons 2 and 3 respectively, shown in FIG. 3A). For PATE and PATE-M genes, 1-2 additional exons, designated 1a and 1b are present between exons 1 and 2, and code for a small number of amino acids. The upstream ACRV1 gene shows a similar exon structure to the PATE-like genes.

All splice events in the PATE-like genes use a +1 phase (FIG. 3A) and, thus alternative splicing involving exon skipping will not alter downstream reading frames. Interestingly, PATE-B and PATE-M generate two splice isoforms (FIG. 2). Cloning and sequencing of the smaller transcripts confirmed that they derive from exon 2 skipping. Both isoforms comprise the putative N-terminal signal peptide but whereas the larger transcript codes for proteins comprising all ten cysteine residues, the smaller transcript codes only for cysteines #6410. The generation of a PATE-M protein that comprises exons 1a+3 is demonstrated in FIG. 3B. The dotted line represents the splice that skips over exon 2 generating a protein coded by exon 1, exon 1a, and exon 3. Removal of the signal peptide coded for by exon 1, will generate the PATE-M protein coded by exons 1a, and exon 3 that comprises five cysteines.

Prediction of signal peptides at the N-terminus of all human PATE-like genes As expected of signal peptide sequences, analysis of all human PATE-like genes reveals clustering of hydrophobic amino acid residues just distal to the initiating methionine (Table 1). Analysis by the SignalP (signal peptide prediction) algorithm predicts with very high probability a signal peptide and accompanying cleavage site for each PATE-like protein (Table 1). That this N-terminal region is in fact a signal peptide directing protein for secretion has been functionally demonstrated for PATE (14) and ACRV1 (SP10) (15) and it is very likely that these regions serve the same function in the PATE-B, PATE-DJ and PATE-M proteins.

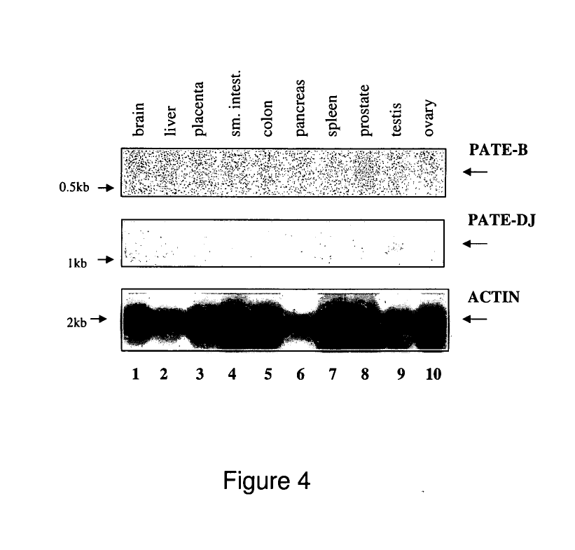

Northern blot analysis of PATE-B and PATE-M expression To extend and confirm the RT-PCR analyses, Northern blot analyses of PATE-B and PATE-DJ were performed (FIG. 4). This demonstrated exclusive expression, albeit at low levels, of PATE-B and PATE-DJ in prostate and testis, respectively, confirming the RT-PCR analyses; no other tissues expressed these genes.

Additional vestigial inactive PATE-like genes in the human PATE-like gene cluster Searches for additional human PATE-like genes within the human gene cluster revealed two potential PATE-like genes, designated PATE-A and PATE-C (FIG. 1). Extensive RT-PCR analyses using a number of forward and reverse primers based on these genomic sequences failed, in all tissues examined, to reveal expression of these genes.

### Example 2

### Identification of Fourteen Active Mouse Pate-Like Genes within the Mouse Genome Synthetic to the Human PATE-Like Genomic Locus

The human PATE-like gene locus extends from the ACRV1 gene to the PATE-B gene, encompassing about 180 kbp. The human genes adjacent to the PATE-like gene locus are (centromeric→telomeric, FIG. 1A): PKNOX2, FEZ1, PIG8, ITM1, CHEK1, ACRV1, (PATE-A), (PATE-C), PATE, PATE-M, PATE-DJ, PATE-B, DDX25 and CDON—this region stretches for about 700 kbp. The syntenic mouse genomic segment was identified and demonstrated the following arrangement: (telomeric→centromeric, FIG. 1B): Pknox2, FezI, Pig8, Itm1, Chk1, Acrv1 (sp10), Svs7 [seminal vesicle secretion protein 7 (16) also called caltrin (17), identified here as the mouse Pate-B ortholog], Ddx25 and Cdon (FIG. 1). Further analyses designed to identify mouse orthologs revealed the mouse Pate-A, Pate-C, Pate, Pate-M, Pate-DJ, and Pate-B (sys7) genes (FIG. 1B). An additional two potential mouse Pate-like genes, designated Pate-E and Pate-N, were identified between Pate-C and Pate. Unexpectedly, inspection of the mouse genomic Pate-like gene locus revealed 0.8 Mbp of genomic DNA situated between the mouse Acrv1 and Pate-A genes. Analysis of this segment revealed several additional Pate-like genes (FIG. 1 and Table 1). The expected human equivalent to the 0.8 Mbp mouse fragment was completely absent.

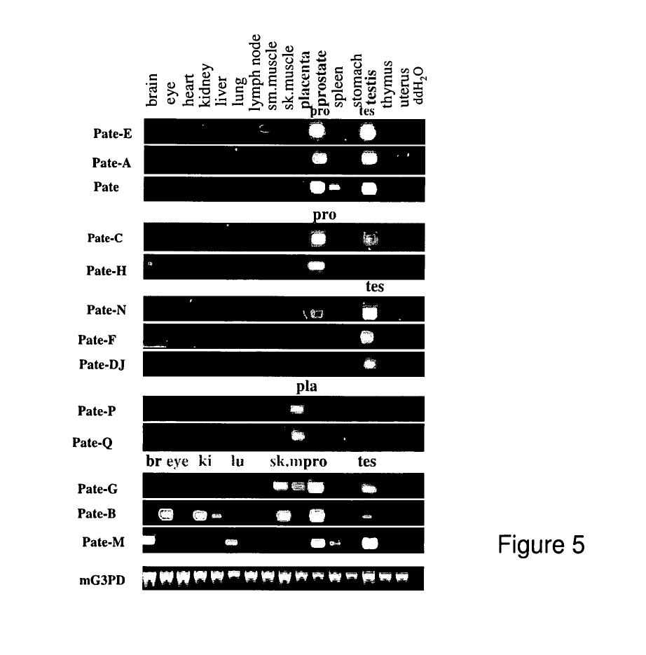

All mouse Pate-like genes code for putative mouse Pate-like proteins comprising a hydrophobic N-terminal signal peptide (Table 1). A multiplex RT-PCR analysis (FIG. 5) utilizing oligonucleotide primers located in exons 2 and 3 revealed that the Pate-A and Pate-C mouse genes (corresponding to the inactive human PATE-C and PATE-A genes) were clearly expressed, in addition to expression of the mouse gene orthologs Pate-B, Pate-DJ, Pate-M and Pate. Even more striking was the discovery of additional seven transcriptionally active mouse Pate-like genes, designated Pate-E, Pate-H, Pate-N, Pate-F, Pate P, Pate-Q and Pate-G (FIGS. 1, and 5, Table 1).

Constituent Pate-like genes could be segregated according to their tissue expression profiles (FIG. 5). Genes Pate-E, Pate-A and Pate were predominantly expressed in both prostate and testis, whereas Pate-C and Pate-H expression was limited to prostatic tissue. Pate-N, Pate-F and Pate-DJ showed almost exclusive expression in testis: Pate-G, Pate-B and Pate-M all showed major expression in either prostate or testis but, in addition, showed significant expression levels in skeletal muscle (Pate-G), eye, kidney and skeletal muscle (Pate-B), and brain and lung (Pate-M). Several mouse Pate-like pseudogenes were scattered amongst the active mouse Pate-like genes (FIG. 1).

Expression in placental tissue of two mouse Pate-like genes Two murine Pate-like genes, Pate-P and Pate-Q, were expressed exclusively in the female-restricted organ, mouse placenta (FIG. 5). Notably, these genes (a) are genomically adjacent to each other (FIG. 1), (b) code for highly similar proteins (Table 1) and (c) both comprise an eleventh cysteine residue in addition to the consensus Pate-like ten cysteine residues. Supporting placental expression reported here, is a trophoblast cDNA library EST (BQ032923) representing a partial Pate-Q sequence.

Castration induces Pate-like gene expression in the ventral prostate that is ablated by subsequent dihydroxy-testosterone administration The selective tissue distribution of Pate-like gene expression in the testis, prostate and placenta (and pregnant and lactating mammary gland, see below) indicated that the Pate-like proteins are likely involved in reproductive-related behavior.

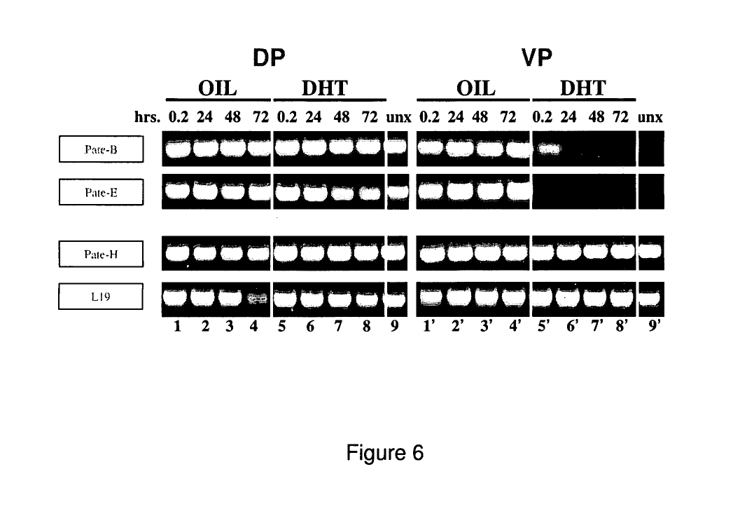

To see whether in-vivo hormonal changes may affect Pate-like gene expression, we investigated the effects of castration and subsequent dihydroxytestosterone (DHT) administration on prostate expression of the Pate-like genes. As differential gene expression has been documented in the anatomically discrete dorsal and ventral prostate lobes, we investigated Pate-like gene expression in each separate lobe. The dorsal lobe in uncastrated mice clearly expressed the two Pate-like genes, Pate-B and Pate-E (FIG. 6, lane 9). Dorsal lobe expression remained high, irrespective of castration and subsequent DHT administration (FIG. 6, lanes 1-4 and 5-8, respectively). In contrast to dorsal lobe expression, the Pate-like genes were not expressed in the uncastrated ventral lobe (FIG. 6, lane 9′). However, following castration Pate-B and Pate-E were clearly expressed in the ventral lobe (FIG. 6, lanes 1′-4′). DHT administration subsequent to castration ablated this expression (FIG. 6, lanes 5′-8′). The DHT-mediated suppression of ‘castration-induced ventral lobe expression’ occurred swiftly (FIG. 6, lane 5′)—fifteen minutes of DHT administration completely extinguished Pate-E expression and led to the partial suppression of Pate-B expression that was complete following 24 hours DHT treatment. In contrast to the differential dorsal and ventral lobe expression of Pate-B and Pate-E, the Pate-H gene demonstrated high expression in both dorsal and ventral lobes (FIG. 6, lanes 9 and 9′, respectively). Neither castration nor subsequent DHT treatment altered this expression (FIG. 6, lanes 1-8 and l′-8′). In fact, the Pate-H gene universal expression served as a convenient internal control for integrity of prostate cDNAs as did the housekeeping L19 gene (FIG. 6 lanes 1-9 and 1′-9′).

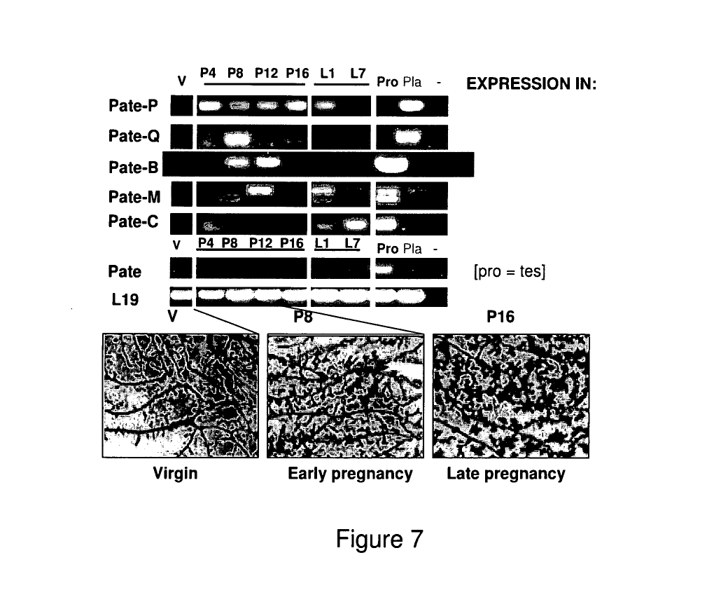

Pate-like gene expression in mammary glands of pregnant and lactating mice The effect of hormonal changes on Pate-like gene expression in female mice was also examined. The hormonally-regulated mouse mammary gland served as a convenient female tissue to address this question. No Pate-like genes were expressed in virgin mammary gland tissue (FIG. 7, lane V). In contrast, mammary glands derived from either pregnant or lactating mice demonstrated expression of Pate-C, -P, -Q, -B and -M. Pregnant mammary gland tissue predominantly expressed Pates-P, Q, B and M. Pate-P showed highest expression levels on days 4 and 16 of pregnancy (FIG. 7, P4 and P8), Pate-Q on day 8 (FIG. 7, P8) and Pate-B on day 12 (FIG. 7, P12). Pate-M showed expression primarily on P12 with significantly lower levels on P8 (FIG. 7, P12 and P8)—Pate-M was also expressed on day 1 of lactation (FIG. 7, L1). Interestingly, expression of Pate-M isoforms varied at different days of pregnancy and lactation-smaller isoform (without exon 1a, see Table 1, for Pate-M Exon 1a) at P8, larger isoform at P12, and predominantly larger isoform at L1 (FIG. 7). Pate-C was expressed almost exclusively on day 7 of the lactating mammary gland—this expression pattern tallies with the mRNA NM_{—}026593 (corresponding to Pate-C) found predominantly in mouse prostate and lactating mammary gland cDNA libraries. Of all the 14 mouse Pate genes that we had identified, only the 5 Pate-like genes described here displayed expression in either the pregnant or lactating mammary gland. The remaining genes, such as Pate for example (FIG. 7, Pate), were not expressed in these tissues.

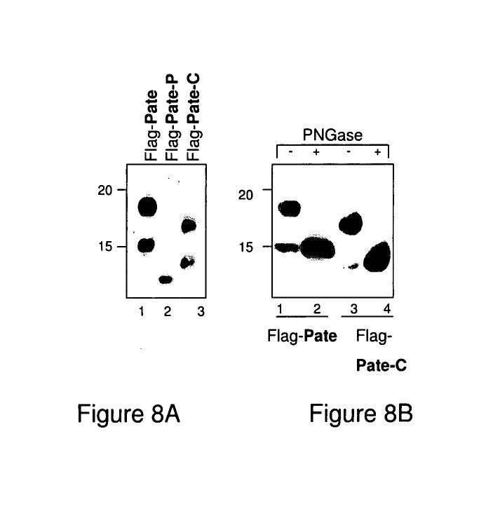

Analysis by SDS PAGE of Pate-like proteins To visualize the Pate-like proteins and to see whether besides formation of disulfide bonds they may undergo additional post-translational modifications, mouse Pate, Pate-C and Pate-P were expressed in a mammalian cell expression system. Western blot analysis revealed (FIG. 8) that all three Pate-like proteins were clearly expressed in mammalian cells. Furthermore, whereas the Pate-P protein migrated with its expected mobility (FIG. 8A, lane 2), both the Pate and Pate-C proteins displayed two bands (FIG. 8A, lanes 1 and 3, respectively). Treatment with PNGaseF, an enzyme that removes N-linked sugars from protein resulted in increased mobility of the Pate and Pate-C proteins (FIG. 8B, compare lanes 1 and 2, 3 and 4, respectively), demonstrating that both comprise N-linked sugars. The Pate protein showed a single, consensus N-glycosylation site, N-C-T, whereas Pate-C harbors an NS-C sequence, an alternative N-linked glycosylation site (18).

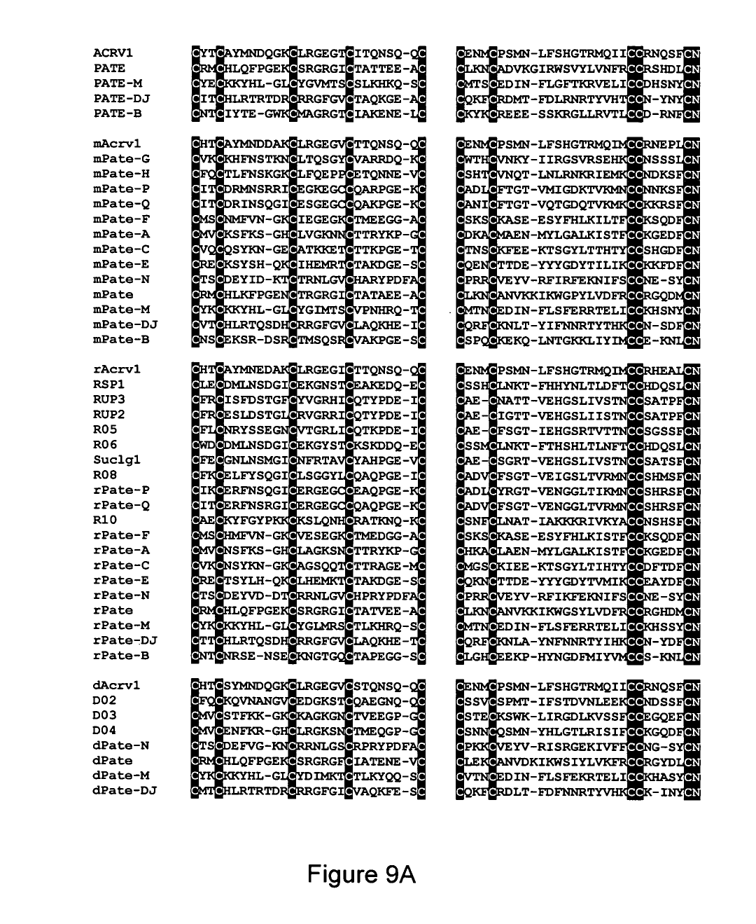

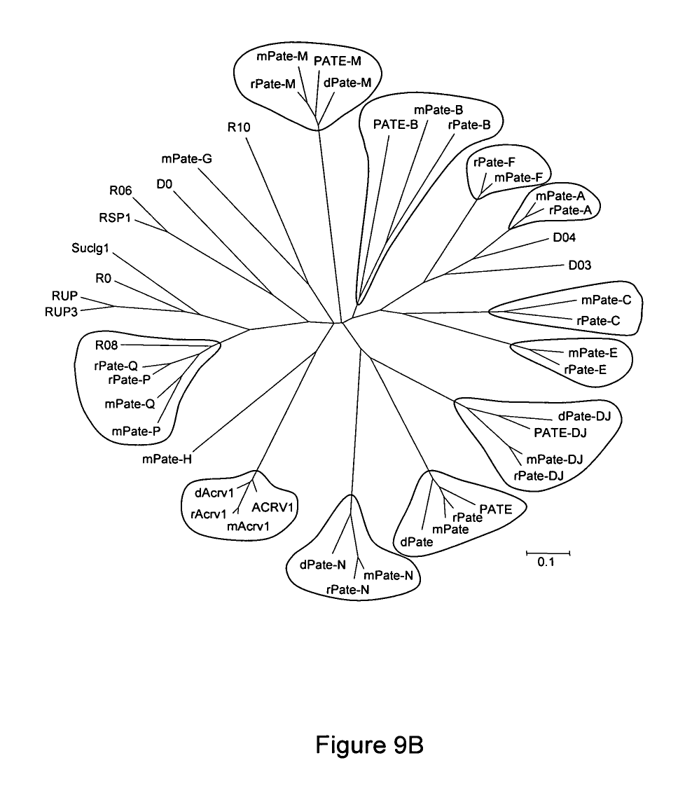

Presence in rat and dog genomes of Pate-like genes Investigation of the rat synthetic locus revealed a 2.5 Mbp insertion, corresponding to that of the mouse 0.8 Mbp insert, located between the rat Acrv1 (sp10) and Pate-A genes. This analysis showed that the mouse Pate-like genes Acrv1, Pate-P, Pate-Q, Pate-F, Pate-A, Pate-C, Pate-E, Pate-N, Pate, Pate-M, Pate-DJ, and Pate-B all have rat orthologs (FIG. 9). We could not assign rat orthologs to the mouse Pate-G and Pate-H genes. The rat locus harbors several Pate-like genes that code for proteins that appear rat-specific, including RSP1 (rat spleen protein 1), RUP2 (rat urinary protein 2), RUP3 (rat urinary protein 3) (19), Suclgl, and an additional five rat Pate-like genes that could not be assigned a mouse ortholog. Analysis of the dog Pate-like gene locus revealed dog (d)Acrv1, dPate, dPate-M and dPate-DJ. Four additional dog Pate-like genes located between dAcrv1 and dPate (FIG. 9) could not be clearly assigned to any human, mouse or rat orthologs.

### Example 3

### Association of Pate-Like and PATE-Like Genes with Energy Homeostasis

Intake of dietary oil modulates, in the intact organism, Pate-like gene expression, thereby demonstrating that the PATE-like genes may play a pivotal role in energy homeostasis.

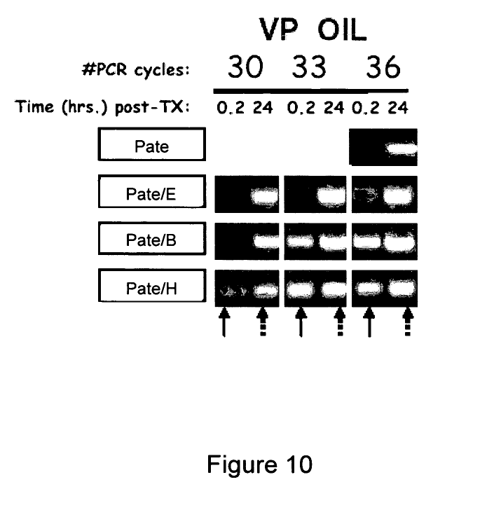

The expression of several Pate-like genes in the ventral prostate was assessed in response to administration of sesame oil to mice. Surprisingly, as can be seen in FIG. 10, the expression of Pate E and Pate B was found to be remarkably upregulated upon administration of the sesame oil.

At the 0 hr time point (tissue extracted immediately following sesame oil injection), no expression of Pate was seen (FIG. 10, 36 PCR cycles, 0 hr time point). In contrast, 24 hours following sesame oil administration, Pate gene expression was clearly induced (FIG. 10, 36 PCR cycles, 24 hr. time point).

Induction of expression was also evident for Pate-E and Pate-B. At the 0 hr time point only very limited Pate-E and Pate-B expression is observed (FIG. 10, 30, 33 and 36 PCR cycles, 0 hr. time point). However, 4 hours post-sesame oil administration significant expression levels are seen (FIG. 10, 30, 33 and 36 PCR cycles, 24 hr. time point).

Analysis of an additional Pate-like gene, Pate-H demonstrates that the Pate-like genes are differentially induced by sesame oil. Pate-H displays relatively high expression levels in the absence of sesame oil (FIG. 10, 30, 33 and 36 PCR cycles, 0 hr. time point), that are only modestly increased following oil administration (FIG. 10, 30, 33 and 36 PCR cycles, 24 hr. time point).

These experiments clearly show that in the intact organism intake of dietary oil, such as sesame oil, modulates Pate-like gene expression, thus establishing a connection between Pate-like gene expression and body energy balance.

Further support for the surprising finding that PATE-like genes play a role in the response to food intake, and thus potentially in energy balance, appetite and body weight gain or loss is provided by the tissue expression pattern of ACRV1. The human ACRV1 gene, that is chromosomally located upstream to human PATE and also belongs to the PATE-like gene cluster, is quite evidently expressed not only in testis, but also in the pancreas, an organ undoubtedly involved in secretion of hormones that regulate energy balance and fat metabolism. As shown in FIG. 2A, human ACRV1 is primarily expressed in testis but also in pancreas. Concordant with this finding, when the human tissues cDNAs were subjected to 40 PCR cycles (FIG. 2B) to increase detection sensitivity, the PATE and PATE-DJ genes were found to be clearly expressed in pancreatic tissue (FIG. 2B, indicated by thick dotted yellow circles) with lower pancreatic expression levels of the PATE-M and PATE-B genes (indicated by thin dotted yellow circles).

Interestingly, ACRV1 EST (expressed sequence tags) were detected in cDNA libraries obtained from medulla oblongata, hippocampus and placenta in addition to many EST deriving as expected from testis cDNA libraries. Additionally, of the 13 human mRNAs and ESTs reported in the UCSC genome Browser for the human PATE gene entry (on chromosome 11q, nucleotides 125,122,000-125,124,500), six are from prostate and three from testis cDNA libraries, as expected-intriguingly the remaining three are from medulla oblongata cDNA libraries. This demonstrates that both ACRV1 and PATE are expressed in the medulla oblongata and ACRV1 is also expressed in the hippocampus. Regulation of energy homeostasis by brain stem (medulla oblongata) and vagal inputs and not only by the classic hypothalamic circuits has been previously demonstrated. We therefore propose that ACRV1 and PATE gene expression in the medulla and hippocampus may be involved with regulation of energy homeostasis.

### Example 4

### Association of Pate-Like and PATE-Like Genes with the Nervous System and Neural Transmission

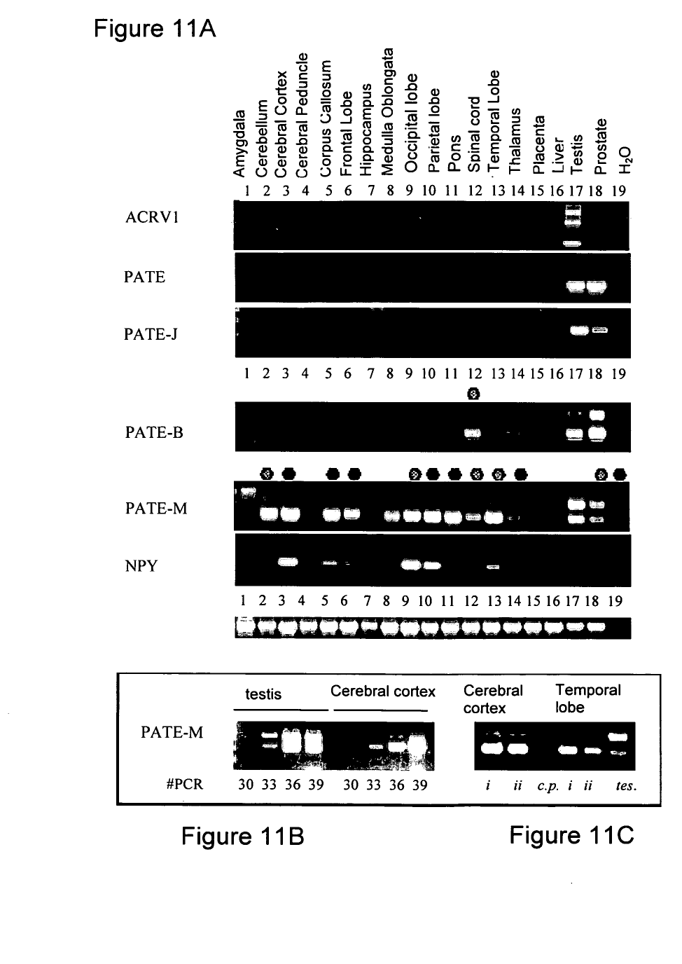

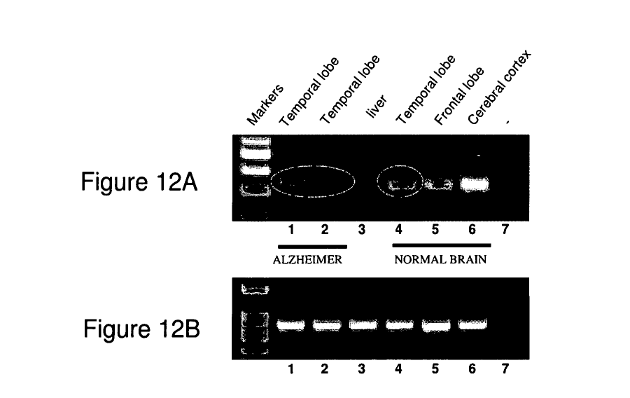

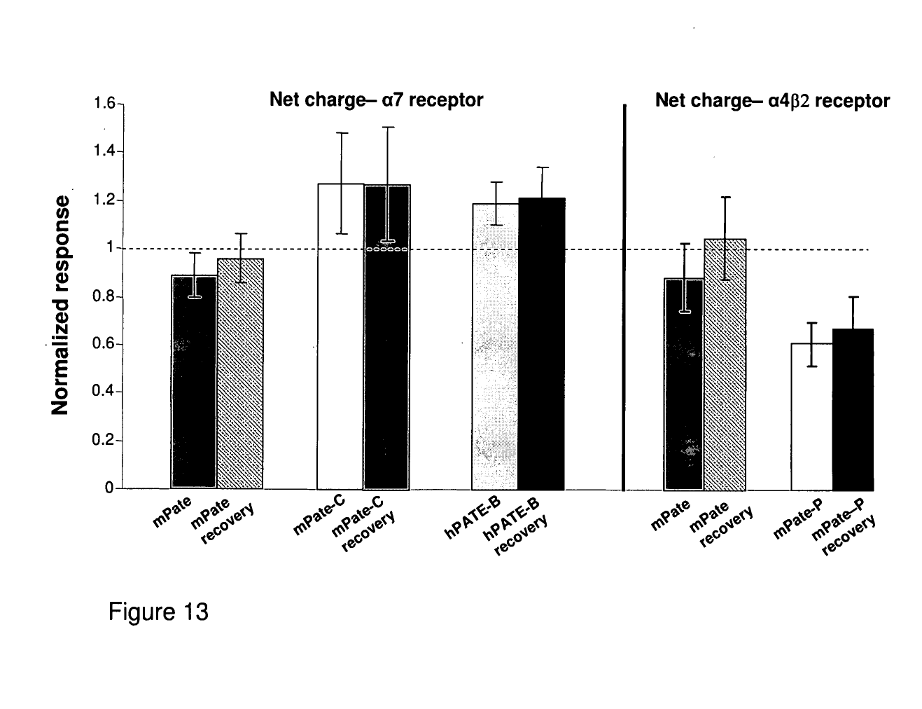

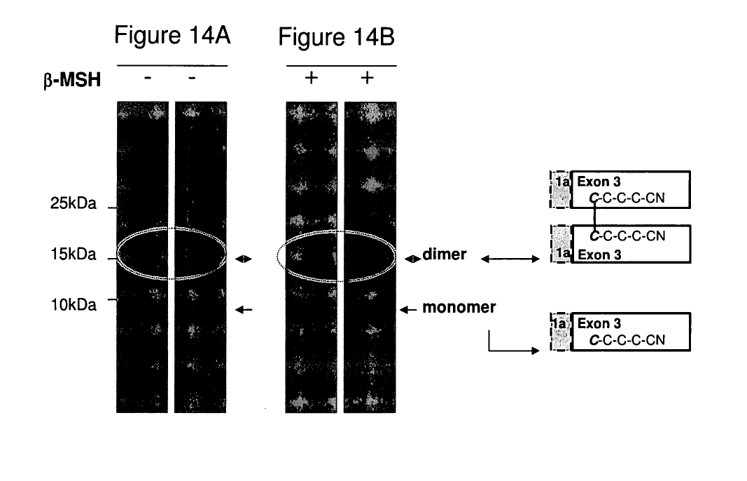

Mouse Pate-M gene was found to be highly expressed in brain tissue in addition to its expression in mouse testis and prostate (FIG. 5, panel Pate-M). Concordant with this finding, analysis of expression of the human PATE-like genes demonstrated high expression of the small alternative PATE-Mₛₘₐₗₗ splice form (comprising exons #1 and #3) in many different regions of the brain, in addition to its expression in testis and prostate (FIG. 11, panel PATE-M), where both PATE-M isoforms, PATE-M and PATE-Mₛₘₐₗₗ, are observed. Human PATE-B is also expressed to significant extents in spinal cord tissue (PATE-B, lane 12), in addition to its expression in prostate and testis. Notably, only the smaller PATE-B isoform (comprising exons #1 and #3) is expressed in spinal cord tissue. In contrast, ACRV1, PATE and PATE-DJ were expressed, as expected, in testis and prostate tissues (lanes 17 and 18, respectively), but no expression was observed in any regions of the brain (lanes 1-14).

Prominent expression of PATE-Mₛₘₐₗₗ was seen in cerebellum, cerebral cortex, corpus callosum, frontal lobe, medulla oblongata, occipital lobe, parietal lobe, pons and temporal lobe (PATE-M, lanes 2, 3, 5, 6, 8-11 and 13). As “neuropeptide Y” (NPY) is the most highly expressed neuropeptide in brain, its expression in the various brain tissues was examined and compared to that of PATE-M. This showed NPY expression in the cerebral cortex, occipital lobe and parietal lobe (NPY, lanes 3, 9 and 10). As compared to NPY, PATE-M was expressed at higher levels and in a wider spectrum of different brain regions. A semi-quantitative comparative analysis of PATE-M expression in testis (the tissue where highest PATE-M expression has to date been detected) and brain cerebral cortex (FIG. 11B) demonstrated similar high PATE-M expression levels in both tissues (signals in both testis and cerebral cortex observed following 33 PCR cycles). In contrast to testis where both PATE-M and PATE-Mₛₘₐₗₗ isoforms are expressed, in cerebral cortex there is almost exclusive expression of PATE-Mₛₘₐₗₗ. The exclusive expression of the PATE-Mₛₘₐₗₗ isoform was seen in two separate samples of cerebral cortex and temporal lobe (FIG. 11C, i and ii). As a control, PATE-M expression in testis (tes.) demonstrated, as expected, expression of both PATE-M and PATE-Kₛₘₐₗₗ isoforms (FIG. 11C, tes.), The very low levels of PATE-M expression seen in the cerebral peduncle sample (c.p), highlight the differential expression of PATE-M in different regions of the brain.

FIG. 12 demonstrates decreased expression of the PATE-M five-cysteine-containing protein in the brains of Alzheimer patients. This finding demonstrates that the level of this PATE-M protein changes in a central nervous system pathology as compared to normal brains, and indicates that administering this protein to such patients may have therapeutic consequences.

### Example 5

### Human PATE(Pate)-Like Proteins Modulate Activity of Nicotinic Acetylcholine Receptors (nAChRs)

Various nAChRs were expressed using the Xenopus oocyte cell expression system, and evoked changes in channel activation that occur in response to application of the recombinant PATE (Pate)-like proteins were assessed. In this study, measurement of net charge accumulation over the entire period of drug administration was evaluated. This analysis produces results essentially identical to those obtained by more complicated concentration-correction methods. The net charge is a particularly attractive parameter as it represents the time integration of all activated channels responding to drug administration.

The α4β2 and α7 nAChRs were expressed in Xenopus oocytes as described in Methods. After obtaining initial control responses to 8 second applications of ACh, cells were washed for four minutes with either control Ringers or Ringers plus the PATE-like proteins (60 nM or 200 nM) and then challenged with ACh or ACh plus the PATE-like proteins, respectively. Responses to ACh plus the PATE-like proteins were normalized to the net charge responses to ACh alone for each oocyte. ACh concentrations were 30 μM for α4β2-expressing cells and 60 μM for α7-expressing cells. To test for recovery, oocytes were then washed for an additional four minutes in the absence of acetylcholine and the PATE-like proteins and then acetylcholine (30 μM or 60 μM) was applied for 8 seconds and the net charge calculated to give the recovery values.

As shown in FIG. 13 application of mouse Pate (mPate) to both α4β2 and α7 nAChRs or human PATE-B (hPATE-B) to the α4β2 nAChR did not evoke any changes in net charge. In contrast, an increase in the net charge was measured after application of hPATE-B to the α7 nAChR, a change that was statistically significant (p<0.05) and persisted even after a four minute wash (FIG. 13). Additionally, mPate-C increased the net charge when applied to human α7 nAChRs, a statistically significant change that also persisted after a four minute wash (FIG. 13). Interestingly, the mPate-P protein did not elicit any changes when applied to the human α7 nAChRs, but did evoke a statistically significant decrease in net charge when added to the α4β2 nAChR, a change that also persisting after a four minute wash (FIG. 13). In summary, of the four assayed recombinant PATE (Pate)-like proteins, three (hPATE-B, mPate-C and mPate-P) showed modulatory activity of the nAChRs.

### Example 6

### Dimerization of Human PATE-M 5-Cysteine Protein (Exons 1a+3)

The following example provides experimental evidence for the ability of the PATE-like proteins that contain five cysteine residues to dimerize.

A recombinant protein encoded by PATE-M exons [1a+3] and tagged at its C-terminus with six histidine residues was produced in IPTG-induced BL21 Origami bacteria.

The protein was purified from inclusion bodies by guanidinium hydrochloride solubilization, dialysis, and nickel-agarose purification. Aliquots were electrophoresed through a 16.5% SDS-PAGE-Tricine gel under non-reducing or reducing conditions in the absence (−) or presence of β-mercaptoethanol (β-MSH), immunoblotted onto a PVDF membrane and probed with anti-histidine tag rabbit polyclonal antibodies followed by ECL detection. As shown in FIG. 14, samples that were electrophoresed under non-reducing conditions demonstrated the presence of a large protein band corresponding to the PATE-M dimer as indicated by its size. When the samples were electrophoresed under reducing conditions which cause the opening of cysteine bonds an additional protein band becomes apparent. This band represents the monomer as indicated by its size.

This example emphasized the ability of the small PATE variant to form dimeric molecules.

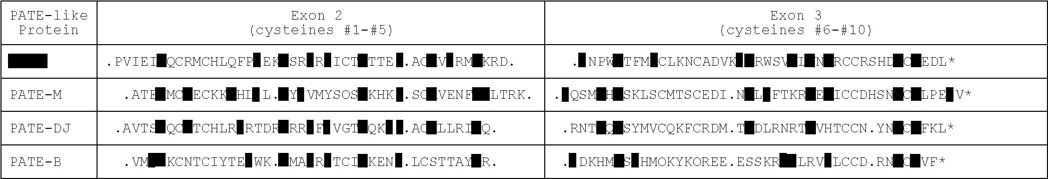

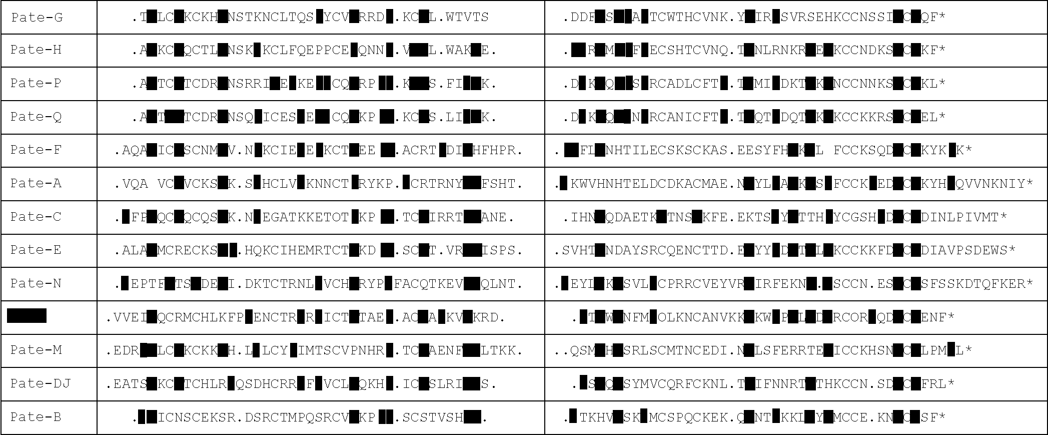

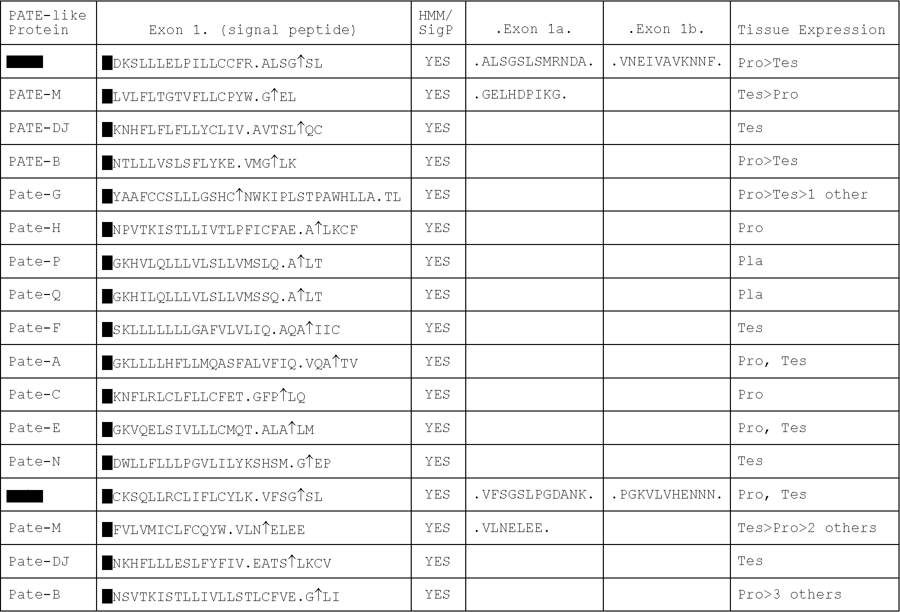

1. An isolated polynucleotide encoding for a PATE-like protein comprising a sequence selected from the group consisting of
SEQ ID No. 1 (encoding for PATE-DJ),
SEQ ID No. 5 (encoding for PATE-B exons 1 and 3),
SEQ ID No. 4 (encoding for PATE-M exons 1, 2 and 3),
SEQ ID No. 6 (encoding for PATE-M exons 1, 1a and 3),
SEQ ID No. 7 (encoding for PATE-M exons 1 and 3),
and a polynucleotide having a degree of identity of at least about 70%, preferably at least about 80%, more preferably at least about 85%, also more preferably at least about 90%, and most preferably at least about 95%.

2. An isolated PATE like polypeptide comprising an amino acid sequence selected from the group consisting of
SEQ ID No. 8 (PATE-DJ),
SEQ ID No 12 (PATE-B “1, 3”),
SEQ ID No 11 (PATE-M “1, 2, 3”),
SEQ ID No 13 (PATE-M “1, 1a, 3”),
SEQ ID No. 14 (PATE-M “1, 3”),
and a polypeptide having a degree of identity of at least about 70%, preferably at least about 80%, more preferably at least about 85%, also more preferably at least about 90%, and most preferably at least about 95%.

3. An isolated multimeric polypeptide comprising at least two short variant PATE-like polypeptides conjugated via cysteine-cysteine bonds, wherein the short variant PATE-like polypeptides are selected from the group consisting of SEQ ID No. 12, SEQ ID No. 13, SEQ ID No. 14 and a short variant PATE-like polypeptide having a degree of identity of at least about 70%, preferably at least about 80%, more preferably at least about 85%, also more preferably at least about 90%, and most preferably at least about 95% to SEQ ID No. 12, 13 or 14, and wherein the short variant PATE-like polypeptides having 5 cysteine residues.

4. The isolated multimeric polypeptide according to claim 3, wherein the multimeric protein is a homodimer or heterodimer protein comprising two short variant PATE-like polypeptides conjugated via cysteine-cysteine bonds.

5. The isolated homodimer according to claim 4, comprising two PATE B short variant polypeptides of SEQ ID NO. 12.

6. The isolated homodimer according to claim 4, comprising two PATE M short variant polypeptides of SEQ ID NO. 13 or SEQ. ID NO 14.

7. The isolated heterodimer according to claim 4, comprising one PATE B short variant polypeptide of SEQ ID NO. 12 and one PATE M short variant polypeptide of SEQ ID NO. 13 or 14.

8. The isolated multimeric polypeptide according to claim 3, wherein at least one of the short variant PATE-like polypeptides is an ACRV1ₛₘₐₗₗ polypeptide.

9. A method of treating a disease or disorder, comprising: administering to a patient in need thereof a therapeutically effective amount of a pharmaceutical composition comprising a compound selected from the group consisting of
a molecule that interacts with a PATE-like polypeptide;
an antibody capable of specifically binding to an epitope of a PATE-like polypeptide;
a PATE-like polypeptide; and
an agent that affects the synthesis or the secretion of PATE-like polypeptides from cells;
wherein the PATE-like polypeptide being in accordance with claim 2.

10. The method according to claim 9, wherein the disease or disorder is selected from a group consisting of:
a disease or disorder associated with the reproductive system;
a disease or disorder associated with prostate or testis;
a disease or disorder associated with body energy homeostasis, appetite or food intake; and
a disease or disorder associated with the central nervous system.

11. The method according to claim 10, wherein the disease or disorder is Alzheimer's disease.

12. The method according to claim 10, wherein the disease or disorder is associated with nicotinic acetylcholine receptors.

13. A method of modulating the activity of nicotinic acetylcholine receptors (nAChR), comprising: administering a therapeutically effective amount of at least one isolated PATE-like polypeptide in accordance with claim 2, to cells expressing the nAChR.

14. A method according to claim 13, wherein the modulating the activity of nicotinic acetylcholine receptors comprises increasing the net charge of α7 nAChR by administration of at least one isolated PATE-like polypeptide comprising an amino acid sequence selected from the group consisting of
SEQ ID No. 8 (PATE-DJ),
SEQ ID No 12 (PATE-B “1, 3”),
SEQ ID No 11 (PATE-M “1, 2, 3”),
SEQ ID No 13 (PATE-M “1, 1a, 3”),
SEQ ID No. 14 (PATE-M “1, 3”),
and a polypeptide having a degree of identity of at least about 70%, preferably at least about 80%, more preferably at least about 85%, also more preferably at least about 90%, and most preferably at least about 95% to cells expressing said the nAChR.

15. The method according to claim 14, wherein the at least one isolated PATE-like polypeptide in is administered in a concentration of between about 10 nM and about 300 nM.

16. The method according to claim 15, wherein the at least one isolated PATE-like polypeptide in is administered in a concentration of between about 50 nM and about 250 nM.

17. The method according to claim 13, wherein the isolated PATE-like polypeptide is human PATE-B.

18. The method according to claim 13, wherein the modulation is performed in vitro.

19. A pharmaceutical composition comprising as an active ingredient a compound selected from the group consisting of
a molecule that interacts with a PATE-like polypeptide;
an antibody capable of specifically binding to an epitope of a PATE-like polypeptide;
a PATE-like polypeptide in accordance with claim 2; and
an agent that affects the synthesis or the secretion of PATE-like polypeptides from cells;
and a pharmaceutically acceptable carrier.

20. The pharmaceutical composition according to claim 19, for the treatment of a disease or disorder selected from the group consisting of
a reproductive-system related condition;
body energy homeostasis related conditions;
obesity; and
disorders of the nervous system.

21. The pharmaceutical composition according to claim 20, for the treatment of neural-transmission related conditions.

22. The pharmaceutical composition according to claim 20, wherein the disease or disorder is Alzheimer's disease.

23. The pharmaceutical composition according to claim 20, wherein the disease or disorder is associated with nicotinic acetylcholine receptors.

24. A method of diagnosing a nervous system disease or disorder, comprising:
obtaining a sample of nervous system tissue;
preparing mRNA from said the sample; and
assessing the expression level of PATE-like mRNA in the sample;
wherein a decreased level of mRNA expression compared with normal nervous system tissue is indicative of a nervous system associated disease.

25. The method according to claim 24, wherein the nervous system tissue is brain tissue.

26. A method of diagnosing a disease or disorder, comprising:
obtaining a sample of a subject's body fluid;
contacting the sample of body fluid with an antibody capable of specifically binding to an epitope of a PATE-like polypeptide;
assessing the level of the PATE-like polypeptide in the sample;
wherein the PATE-like polypeptide is selected from the group consisting of SEQ. ID No. 8, 12, 13 and 14; and wherein a decreased or increased level of the PATE-like polypeptides compared to the level in normal subjects is indicative of a disease or disorder.

27. The method according to claim 24 wherein the disease or disorder is Alzheimer's disease.
